(19) United States
(12) Patent Application Publication
(10) Pub. No.: US 2010/0298219 A1
(43) Pub. Date: Nov. 25, 2010

(54) METHOD OF TREATING CANCER, NEOPLASTIC DISORDERS, AND SYMPTOMS THEREOF WITH COMPOUNDS

(76) Inventors: Anthony Cerami, Ossining, NY (US); Michael Brines, Woodbridge, CT (US)

Correspondence Address: JONES DAY, 222 EAST 41ST ST, NEW YORK, NY, 10017, US

(21) Appl. No.: 12/863,915

(22) PCT Filed: Jan. 22, 2009
(86) PCT No.: PCT/US09/00466

§ 371 Date: Jul. 21, 2010

(51) Int. Cl. A61K 38/18; A61P 35/00; A61P 35/02; A61P 35/04

(52) U.S. Cl. 514/7.7

(57) ABSTRACT

The present invention is directed to the use of tissue protective compounds for the prevention, treatment, amelioration or management of cancer, neoplastic diseases, their symptoms and side-effects associated with the treatment of these indications, i.e. chemotherapy or radiation therapy. In particular, these compounds may be chemically modified erythropoietin peptides or recombinant derivatives of erythropoietin, and preferably these compounds lack erythropoietic activity or have substantially reduced erythropoietic activity.

### 1. INTRODUCTION

This invention encompasses methods and uses of compounds to treat, prevent or ameliorate cancer, a neoplastic disease, symptoms thereof, i.e. cachexia, or side effects associated with current treatments for cancer, i.e. chemotherapy or radiation. In particular, compounds useful within the current method exhibit tissue protective activity such as neuroprotection, neurotrophic activities, or anti-apoptotic activities in addition to their ability to arrest or retard the growth of tumors, benign or cancerous. Further, these compounds preferably lack erythropoietic or hematopoietic activity.

### 2. BACKGROUND OF THE INVENTION

Tumors are abnormal growths, and are characterized as being benign or cancerous. Benign tumors, are growths that lack the malignant properties of a cancerous tumor, and are generally mild and nonprogressive tumors. Moles and uterine fibroids are examples of benign tumors. However, in some instances neoplasms which are defined as ‘benign tumors’ because they lack the invasive properties of a cancer, may still produce negative health effects. Examples of this include tumors which produce a “mass effect” (compression of vital organs such as blood vessels), or “functional” tumors of endocrine tissues, which may overproduce certain hormones (examples include thyroid adenomas, adrenocortical adenomas, and pituitary adenomas). Furthermore, although the benign tumor may lack malignant characteristics, many types of benign tumors have the potential to become malignant and some types, such as teratoma, are notorious for this.

Cancerous tumors are abnormal growths caused by abnormalities in the genetic material of cancerous cells. These abnormalities may be due to the effects of carcinogens, such as tobacco smoke, radiation, chemicals, or infectious agents (viruses such as Hepatitis C). Cancerous growths may also be triggered by random errors in DNA replication, or may be inherited, and thus present in all cells from birth. The resulting genetic abnormalities found in cancer typically affect two general classes of genes: activation of oncogenes, which give the cancer cells new properties, such as hyperactive growth and division, protection against programmed cell death, loss of respect for normal tissue boundaries, and the ability to become established in diverse tissue environments; and/or the inactivation of tumor suppressor genes which results in the loss of normal functions in those cells, such as accurate DNA replication, control over the cell cycle, orientation and adhesion within tissues, and interaction with protective cells of the immune system. Cancerous growths are distinguished form other growths such as benign tumors by several malignant properties: their ability (1) to grow and divide without respect to normal limits, (2) to invade and destroy adjacent tissues, and (3) to sometimes spread to other locations in the body). Cancer can affect people of all ages, and it is estimated that 7.6 million people died of cancer in 2007 worldwide. [cite] In the United States Alone 559,650 people died from cancer, and an estimated 1,444,920 people were diagnosed with cancer in 2007. (National Cancer Institute).

Currently cancer therapy involves surgery, chemotherapy, and/or radiation treatment to eradicate neoplastic cells in a patient (see for example, Stockdale, 1998, “Principles of Cancer Patient Management,” in Scientific American: Medicine, vol. 3, Rubenstein and Federman, eds., Chapter 12, Section N). All of these approaches pose significant drawbacks for the patient. Surgery, for example, may be contraindicated due to the health of the patient or may be unacceptable to the patient. Additionally, surgery may not completely remove the neoplastic tissue given the ability of a single cancerous cell to metasticize and relocate elsewhere in the body. Additionally, the surgery may adversely impact the quality of life of the patient, i.e. removal of a prostate tumor may result in loss of sexual function or removal of an optical tumor may result in blindness in the affected eye. Radiation therapy is effective only when the irradiated neoplastic tissue exhibits a higher sensitivity to radiation than normal tissue, and radiation therapy can often elicit serious side effects. (Id.) With respect to chemotherapy, there are a variety of chemotherapeutic agents available for the treatment of neoplastic disease. However, despite the availability of a variety of chemotherapeutic agents, chemotherapy has many drawbacks. Almost all chemotherapeutic agents are toxic and chemotherapy causes significant and often dangerous side effects, including severe nausea, diarrhea, bone marrow depression, immunosuppression, etc. Additionally, many tumor cells are resistant to chemotherapeutic agents through multi-drug resistance.

In an effort to address the significant need for new therapies to treat or prevent cancer or neoplastic diseases with less severe or negligible side-effects researchers have explored many alternative therapies, including the use of various cytokines. Erythropoietin, a Type-1 cytokine, has been targeted as a potential cancer therapeutic. Erythropoietin (“EPO”) is a glycoprotein hormone commonly associated with the maintenance of hematocrit and, more recently, tissue protection. Mature human EPO protein comprises 165 amino acids and has a molecular weight of about 30.4 kDa measured by mass spectroscopy, with glycosyl residues contributing about 40% of the weight of the molecule. The EPO molecule comprises four helices that interact via their hydrophobic domains to form a predominantly globular structure within an aqueous environment (Cheetham et al., 1998, Nat. Struct. Biol. 5:861-866, which is hereby incorporated by reference in its entirety).

As noted above, EPO is pluripotent. In its hormonal role, EPO regulates hematocrit through its role in the maturation of erythroid progenitor cells into erythrocytes and therefore is used to treat anemia in patients. Anemia may result from chronic conditions, such as cancer, or treatments associated with these illnesses, such as chemotherapy, which directly suppress the production of EPO. Thus, commercially available recombinant erythropoietin has been available under the trademarks of PROCRIT, available from Ortho Biotech Inc., Raritan, N.J., and EPOGEN, available from Amgen, Inc., Thousand Oaks, Calif. and has been used to treat anemia in oncology patients and those receiving chemotherapy. Based upon the observations that EPO used in this therapeutic indication not only rectified the anemia but resulted in an enhancement of the well being of the patients, researches began to explore the therapeutic capabilities of EPO in addressing the tumors themselves. (See U.S. Pat. No. 6,579,525 and Blau, Erythropoietin in Cancer: Presumption of Innocence?, Stem Cells 2007; 25:2094-2097) U.S. Pat. No. 6,579,525 to Haran-Ghera et al. relates to the use recombinant human EPO for the treatment of multiple myelomas and hypothesizes that EPO induces an immune response to the tumor. Additionally, U.S. patent application Ser. No. 11/093,177, publication no. US 2005/0267027 discloses the use of EPO to inhibit angiogenesis in tumors by reducing HIF-1α and/or VEGF expression in the tumors.

However, the potential of EPO as a cancer therapeutic has not been attained within the clinic. It has been determined that several types of cancer, such as breast cancers, express, and tend to over-express, erythropoietin receptors. This has led to concerns that the therapeutic use of EPO to treat cancer would lead to further growth of the tumor as opposed to a regression of the tumor's development. (See Blau, and U.S. patent application Ser. No. 10/432,899, published as US 2005/0260580). This concern has been bourne out in the clinic as several trials of EPO within various cancer indications have been halted due to an increase in mortality due to tumor growth. (Blau) In light of these adverse clinical outcomes the FDA has attached a Black Box warning on approved EPO products cautioning against their use in nonapproved cancer indications.

### 3. SUMMARY

The present invention is drawn to a method of preventing, treating, ameliorating or managing cancer or neoplastic disorders in a patient in need thereof by administering an effective amount of an isolated compound derived from erythropoietin or another Type-1 Cytokine.

In one embodiment the compounds used to prevent, treat, ameliorate or manage cancer or a neoplastic disorder in a subject in need thereof by are chemically modified erythropoietins selected from the group of i) an erythropoietin that lacks sialic acid moieties, ii) an erythropoietin having at least no sialic acid moieties; iii) an erythropoietin having at least no N-linked or no O-linked carbohydrates; iv) an erythropoietin having at least a reduced carbohydrate content by virtue of treatment of native erythropoietin with at least one glycosidase; v) an erythropoietin having at least one or more oxidized carbohydrates; vi) an erythropoietin having at least one or more oxidized carbohydrates and is chemically reduced; vii) an erythropoietin having at least one or more modified arginine residues; viii) an erythropoietin having at least one or more modified lysine residues or a modification of the N-terminal amino group of the erythropoietin molecule; ix) an erythropoietin having at least a modified tyrosine residue; x) an erythropoietin having at least a modified aspartic acid or a glutamic acid residue; xi) an erythropoietin having at least a modified tryptophan residue; xii) an erythropoietin having at least one amino group removed; xiii) an erythropoietin having at least an opening of at least one of the cystine linkages in the erythropoietin molecule; or xiv) a truncated erythropoietin. In a preferred embodiment, the chemically modified erythropoietin comprises carbamoylated erythropoietin.

In a further embodiment, the compound used to prevent, treat, ameliorate or manage cancer or a neoplastic disorder in a subject in need thereof is a mutated erythropoietin selected from the group of one or more of the following mutations C7S, R10I, V11S, L12A, E13A, R14A, R14B, R14E, R14Q, Y15A, Y15F, Y15I, K20A, K20E, E21A, C29S, C29Y, C33S, C33Y, P42N, T44I, K45A, K45D, V46A, N47A, F48A, F48I, Y49A, Y49S, W51F, W51N, Q59N, E62T, L67S, L70A, D96R, K97D, S100R, S100E, S100A, S100T, G101A, G101I, L102A, R103A, S104A, S104I, L105A, T106A, T106I, T107A, T107L, L108K, L108A, S126A, F142I, R143A, S146A, N147K, N147A, F148Y, L149A, R150A, G151A, K152A, L153A, L155A, C160S, I6A, C7A, B13A, N24K, A30N, H32T, N38K, N83K, P42A, D43A, K52A, K97A, K116A, T132A, I133A, T134A, K140A, P148A, R150B, G151A, K152W, K154A, G158A, C161A, and/or R162A.

In certain aspects, the isolated compounds lack an erythropoietic activity, e.g., increasing hemoglobin in a recipient. Preferably, the isolated compounds lack other activities including, but not limited to, vasoactive action (e.g., vasoconstriction), hyperactivating platelets, pro-coagulant activities and stimulating proliferation and/or production of thrombocytes and/or erythropoietic-dependent cells (see, Coleman et al., 2006, PNAS 103:5965-5970, hereby incorporated by reference in its entirety). In other aspects, the isolated compounds comprise at least one cellular protective activity. Such cellular protective activity includes, but is not limited to, protecting, maintaining, enhancing or restoring the function or viability of a responsive mammalian cell, tissue, or organ.

In another embodiment, the invention relates to methods of arresting the growth of a cell comprising contacting a cell in need of growth arrestment with an effective amount of a compound.

In another embodiment, the invention relates to methods of causing the death of a cancer or neoplastic cell comprising contacting a cancer or neoplastic cell with an effective amount of a peptide.

In another embodiment, the invention relates to methods of inhibiting blood vessel generation to the cancerous or neoplastic cells or reducing the production of molecules causing mitosis or angiogenesis.

In another embodiment, the invention relates to methods for treating or preventing the side-effects associated with chemotherapy or radiation therapy, comprising administering to a patient in need of such treatment or prevention an effective amount of a compound. Side-effects associated with chemotherapy or radiation therapy include cachexia, low blood count, nausea, diarrhea, oral lesions and alopecia.

In another embodiment, the invention relates to methods for treating or preventing cancer or neoplastic disease in a patient comprising contacting a cancer or neoplastic cell with an effective amount of a peptide.

In another embodiment, the invention relates to methods of treating or preventing cancer or neoplastic disease in a patient comprising administering to a patient in need of such treatment or prevention an effective amount of a compound.

In certain embodiments, the invention relates to the use of the compound for the preparation of a pharmaceutical composition for the prevention, treatment, amelioration or management of cancer or neoplastic disorder in a subject in need thereof.

In certain embodiments, the invention is also directed to pharmaceutical compositions comprising the aforementioned isolated compounds for administration to a subject in need thereof. In specific aspects in accordance with this embodiment, the pharmaceutical composition of the invention further comprises a pharmaceutically acceptable carrier. Such pharmaceutical compositions may be formulated for oral, intranasal, ocular, inhalational, transdermal, rectal, sublingual, vaginal, or parenteral administration, or in the form of a perfusate solution for maintaining the viability of cells, tissues, or organs ex vivo. In related embodiments of the invention the subject is a mammalian animal, preferably a human.

### 4. ABBREVIATIONS AND TERMINOLOGY

### 4.1 Abbreviations

As used herein, the abbreviations for the genetically encoded L-enantiomeric amino acids are conventional and are as follows:

| Amino Acid | One-Letter Symbol | Common Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

### 4.2 Terminology

As used herein, the terms “about” or “approximately” when used in conjunction with a number refer to any number within 1, 5, or 10% of the referenced number.

The term “administered in conjunction with” in the context of the methods of the invention means administering a compound prior to, at the same time as, and/or subsequent to the onset of a disease, disorder, or condition.

The term “amino acid” or any reference to a specific amino acid is meant to include naturally occurring protogenic amino acids as well as non-naturally occurring amino acids such as amino acid analogs. Those skilled in the art would know that this definition includes, unless otherwise specifically noted, naturally occurring protogenic (L)-amino acids, their optical (D)-isomers, chemically modified amino acids, including amino acid analogs such as penicillamine (3-mercapto-D-valine), naturally occurring non-protogenic amino acids such as norleucine and chemically synthesized proteins that have properties known in the art to be characteristic of an amino acid. Additionally, the term “amino acid equivalent” refers to compounds that depart from the structure of the naturally occurring amino acids, but which have substantially the structure of an amino acid, such that they can be substituted within a compound, which retains its biological activity despite the substitution. Thus, for example, amino acid equivalents can include amino acids having side chain modifications or substitutions, and also include related organic acids, amides or the like. The term “amino acid” is intended to include amino acid equivalents. The term “residues” refers both to amino acids and amino acid equivalents. Amino acids may also be classified into the following groups as is commonly known in the art: (1) hydrophobic amino acids: His, Trp, Tyr, Phe, Met, Leu, Ile, Val, Ala; (2) neutral hydrophilic amino acids: Cys, Ser, Thr; (3) polar amino acids: Ser, Thr, Asn, Gln; (4) acidic/negatively charged amino acids: Asp, Glu; (5) charged amino acids: Asp, Glu, Arg, Lys, His; (6) positively charged amino acids: Arg, Lys, His; and (7) basic amino acids: His, Lys, Arg.

As used herein, an “effective amount” includes that amount of a compound sufficient to destroy, modify, control or remove a neoplasm: a benign tumor, a primary, regional or metastatic cancer cell or tissue; delay or minimize the spread of cancer; or provide a therapeutic benefit in the treatment or management of cancer or a neoplastic disorder. An ‘effective amount” also includes the amount of a compound sufficient to result in cancer or neoplastic cell death.

“Erythropoietic activity” means any significant increase in the levels of hemoglobin or hematocrit in a subject. “Little or no erythropoietic activity” means that an increased level of a subject's hemoglobin or hematocrit meets the criteria accepted in the art as an insufficient increase to cause an adverse effect in a subject. “Significantly increased erythropoietic activity” means that a difference in the level of a subject's hemoglobin or hematocrit compared to a control meets the criteria accepted in the art as significant, which may, inter alia, increase the likelihood of hypertension, seizures, and vascular thrombosis.

“Hematopoietic activity” means any significant increase blood cellular components such as erythroid, lymphoid, and myeloid cells. Further, hematopoietic activity refers to whether an isolated compound posses activity selected from vasoactive action (e.g., vasoconstriction), hyperactivating platelets, pro-coagulant activities, and stimulating proliferation or production of thrombocytes or erythropoietin-dependent cells.

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated” or “purified” compound is substantially free of cellular material or other contaminating proteins from the cell or tissue source from which the protein or compound is derived, or substantially free of chemical precursors or other chemicals when chemically synthesized. The language “substantially free of cellular material” includes preparations of a compound in which the compound is separated from cellular components of the cells from which it is isolated or recombinantly produced. Thus, a compound that is substantially free of cellular material includes preparations of compounds having less than about 30%, 20%, 10%, or 5% (by dry weight) of heterologous protein (also referred to herein as a “contaminating protein”). When the compound is recombinantly produced, it is also preferably substantially free of culture medium, i.e., culture medium represents less than about 20%, 10%, or 5% of the volume of the protein preparation. When the compound is produced by chemical synthesis, it is preferably substantially free of chemical precursors or other chemicals, i.e., it is separated from chemical precursors or other chemicals which are involved in the synthesis of the protein. Accordingly such preparations of the compound have less than about 30%, 20%, 10%, 5% (by dry weight) of chemical precursors or compounds other than the antibody of interest. In a preferred embodiment, compounds of the invention are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, a nucleic acid molecule(s) encoding a compound of the invention is isolated or purified.

As used herein, the term “management” includes the provision of one or more beneficial side effects that a patient derives from a compound which, in one embodiment, does not cure the disease. In certain embodiments, a patient is administered a compound to “manage” a disease so as to prevent the progression or worsening of the disease.

As used herein, the term “neoplasm” or “neoplastic” means an abnormal growth of a cell or tissue (e.g., a tumor) which may be benign or cancerous.

The term “preventing a disease, disorder, or condition” means delaying the onset, hindering the progress, hindering the appearance, protection against, inhibiting or eliminating the emergence, or reducing the incidence, of such disease, disorder, or condition. Use of the term “prevention” is not meant to imply that all patients in a patient population administered a preventative therapy will never develop the disease, disorder, or condition targeted for prevention, but rather that the patient population will exhibit a reduction in the incidence of the disease, disorder, or condition. For example, many flu vaccines are not 100% effective at preventing flu in those administered the vaccine. One skilled in the art can readily identify patients and situations for whom preventative therapy would be beneficial, such as, but not limited to, individuals about to engage in activities that may expose them to carcinogens (e.g., soldiers engaging in military operations, asbestos workers, etc.), patients for whom surgery is planned, patients at risk for inherited diseases, disorders, or conditions, patients at risk for diseases, disorders, or conditions precipitated by environmental factors, or portions of the population at risk for particular diseases, disorders, or conditions such as the elderly, infants, or those with weakened immune systems, or those patients with genetic or other risk factors for a disease, disorder, or condition.

As used herein, a “prophylactically effective amount refers to that amount of a compound sufficient to result in the prevention of the recurrence or spread of a cancer. A prophylactically efficient amount can refer to the amount of a compound sufficient to prevent the recurrence or spread of cancer or the occurrence of cancer in a patient, including but not limited to those predisposed to cancer or previously exposed to a carcinogen. A prophylactically effective amount can also refer to the amount of the compound that provides a prophylactic benefit in the prevention of cancer. Further, a prophylactically effective amount with respect to another prophylactic agent means that amount of that prophylactic agent in combination with a compound that provides a prophylactic benefit in the prevention of Cancer. Used in connection with an amount of a compound, the term “prophylactically effective amount” can encompass an amount that improves overall prophylaxis or enhances the prophylactic efficacy of or provides a synergistic affect with another prophylactic agent.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, ape, or a human), and more preferably a human.

As used herein, the term “tissue protective activity” or “tissue protection” refers to the effect of inhibiting or delaying damage or death of a cell, tissue, or organ. Unless otherwise noted, the “delay” in damage or death of a cell, tissue or organ is evaluated relative to a control condition in the absence of a compound of the invention. Tissue protective activity is specific to tissue, cells, and/or organs expressing a tissue protective receptor complex (i.e., a responsive tissue cell, and.or organ, respectively), such as, but not limited to, the tissues of the central nervous system. In specific embodiments, the responsive cells are not erythrocyte progenitor cells.

The term “tissue protective receptor complex” as used herein means a complex comprising at least one erythropoietin receptor subunit and at least one beta common receptor subunit. The tissue protective receptor complex may contain multiple erythropoietin receptor subunits and/or beta common receptor subunits, as well as other types of receptors or proteins. See WO 2004/096148, which is hereby incorporated by reference herein in its entirety.

As used herein, the term “treatment” includes the eradication, removal, modification, or control of primary, regional, or metastatic cancer tissue; and minimizing the delay of the spread of cancer.

### 5. BRIEF DESCRIPTION OF THE FIGURES

FIG. 1 is a graph comparing the relative mass of a cortical tumor implanted in a rat's brain in accordance with the protocol of Lampson et al. Cancer Res 53:176-82:1993 after 21 days. The graph shows that the relative mass of the tumors in the rats treated with saline increased by slightly less than 20 mm² whereas the rats treated with carbamoylated EPO (CEPO) following implantation showed half the increase in relative mass of the tumor. The relative mass of the tumor in rats treated with rhEPO grew to greater than 30 mm².

FIG. 2 is a image of the immunohistochemistry evaluation of the rats' brains using nestin staining, As can be seen, the rhEPO and saline treated rats show large masses of glioma cells, where as the CEPO treated rats show isolated groupings of the glioma cells.

### 6. DETAILED DESCRIPTION OF THE INVENTION

### 6.1 Compounds of the Invention

Any cytokine that exhibits tissue protective capability is contemplated for use with the present invention. Also, any tissue protective cytokine capable of treating, preventing, ameliorating, or managing cancer or a neoplastic disorder is contemplated as well. As used herein, the term “tissue protective cytokines” refer to any cytokine that is a derivative of erythropoietin that possesses the tissue protective activity of erythropoietin. Preferably the tissue protective cytokine lacks at least one or more of erythropoietin's erythropoietic effects. Most preferably, the tissue protective cytokine lacks all of the erythropoietic effects of erythropoietin. For example, this may be accomplished by modifying erythropoietin through chemical or mutational processes that affect its pharmacological attributes (reduction in half-life) or structural ability to bind to the erythropoietin receptor homodimer. Non-limiting examples of suitable tissue protective cytokines for use with the present invention include the tissue protective cytokines disclosed in International Publication No. WO/02053580 and U.S. Patent Publication Nos. 2002/0086816 and 2003/0072737, which are incorporated by reference herein in their entirety.

In addition, the tissue protective cytokines for use with the present invention may include EPO molecules with a modification of at least one arginine, lysine, tyrosine, tryptophan, or cysteine residue or carboxyl groups are also contemplated for use as tissue protective cytokines according to the present invention. These residues may be chemically modified by guanidination, amidination, carbamoylation, trinitrophenylation, acylation (acetylation or succinylation), nitration, or mixtures thereof, as disclosed in International Publication No. WO/02053580.

Thus, the tissue protective cytokine of the present invention may be carbamoylated EPO. Unlike rhu-EPO and selected other modified EPO molecules, carbamoylated EPO does not retain erythropoietic activity and fails to bind with the classic homodimer erythropoietin receptor as is noted in PCT application No. PCT/US04/013099, filed Apr. 26, 2004, hereby incorporated in its entirety. Carbamoylated EPO, however, does advantageously maintain the tissue protective functionality of endogenous EPO. It is believed that the retained tissue protective function of carbamoylated EPO is mediated through its interaction with a tissue protective receptor complex as disclosed in PCT application No. PCT/US04/013099. Thus, carbamoylated EPO may be used to treat, prevent, ameliorate or manage cancers and neoplastic diseases without posing the risk of the erythropoietic and hematopoietic effects of erythropoietin.

Therefore, the tissue protective cytokine of the invention may be a modified EPO with alteration of at least one or more lysine residues or the N-terminal group of the EPO molecule, which for purposes of this application, may also be referred to as “sites”. The modifications may result from the reaction of the lysine residue or N-terminal amino group with an amino-group modifying agent. For example, the generic reaction scheme below is representative of one method to carbamylate proteins, such as EPO:

In another embodiment, one or more lysine residues on an EPO molecule may be carbamoylated by virtue of reaction with a cyanate ion. For example, one or more lysine residues may be modified by incubation with 4-sulfophenylisothiocyanate. In yet another embodiment, one or more lysine residues on the EPO molecule are alkyl-carbamoylated, aryl-carbamoylated, or aryl-thiocarbamoylated with an alkyl isocyanate, an aryl isocyanate, or an aryl-thioisocyanate, respectively. In still another embodiment, one or more lysine residues are alkylated by a reactive alkylcarboxylic or arylcarboxylic acid derivative, e.g., acetic anhydride, succinic anhydride, or phthalic anhydride. The modified lysine residue may also be chemically reduced.

One or more lysine residues may also be carbamoylated by reacting the residue(s) with trinitrobenzenesulfonic acid, or a salt thereof. In yet another embodiment, one or more lysine residues may be modified by reaction with a glyoxal or a glyoxal derivative, e.g., methylglyoxal it 3-deoxyglucosone, to form the corresponding alpha-carboxyalkyl derivatives.

Other methods of carbamoylation may be used in accordance with the present invention. For example, the method disclosed in Plapp et al., J. BIOL. CHEM., 246: 939-945 (1971) is a suitable way of making the carbamoylated EPO according to the present invention. Another example of a method of carbamoylation is discussed in Satake et al, 1990, Biochim. Biophys. Acta 1038:125-9, where six of the lysine residues in erythropoietin were carbamoylated. Further methods of carbamoylating erythropoietin are disclosed in PCT/US05/023505 and US Patent Publication No. 20060135754.

And, as mentioned above, any of the forms of EPO may be used according to the present invention. Thus, as an example: in one embodiment, the EPO molecule subject to carbamoylation is in α form; in another embodiment, the EPO molecule subject to carbamoylation is in β form; and in yet another embodiment, the EPO molecule subject to carbamoylation is asialic.

The carbamoylation process preferably occurs for a period of time sufficient to substantively reduce or completely eliminate erythropoietic activity. In one embodiment, the carbamoylation process is performed for a sufficient time period to remove at least about 90 percent of the lysines and the N-terminal amino acid. In another embodiment, the carbamoylation process is performed for a sufficient time period to remove at least about 95 percent of the lysines and the N-terminal amino acid. In still another embodiment, the carbamoylation process is performed for a sufficient time period to remove 100 percent of the lysines and the N-terminal amino acid. Alternatively, this may be viewed as carbamoylating erythropoietin for a period of time sufficient to carbamylate at least six lysine residues in one embodiment, at least seven lysines in another embodiment, and at least eight lysine residues in another embodiment. The time required for sufficient carbamoylation to occur may vary. For instance, sufficient carbamoylation may occur over a period up to about 6 to 24 hours, up to about 10 to 20 hours, or up to about 16 hours.

The tissue protective cytokines for use with the present invention may also be obtained by limited proteolysis, removal of amino groups, and/or mutational substitution of arginine, lysine, tyrosine, tryptophan, or cysteine residues by molecular biological techniques as disclosed in Satake et al, 1990, Biochem. Biophys. Acta 1038:125-9, which is incorporated by reference herein in its entirety. For example, suitable tissue protective cytokines include at least one or more mutated EPOs having a site mutation at C7S, R10I, V11S, L12A, E13A, R14A, R14B, R14E, R14Q, Y15A, Y15F, Y15I, K20A, K20E, E21A, C29S, C29Y, C33S, C33Y, P42N, T44I, K45A, K45D, V46A, N47A, F48A, F48I, Y49A, Y49S, W51F, W51N, Q59N, E62T, L67S, L70A, D96R, K97D, S100R, S100E, S100A, S100T, G101A, G101I, L102A, R103A, S104A, S104I, L105A, T106A, T106I, T107A, T107L, L108K, L108A, S126A, F142I, R143A, S146A, N147K, N147A, F148Y, L149A, R150A, G151A, K152A, L153A, L155A, C160S, I6A, C7A, B13A, N24K, A30N, H32T, N38K, N83K, P42A, D43A, K52A, K97A, K116A, T132A, I133A, T134A, K140A, P148A, R150B, G151A, K152W, K154A, G158A, C161A, and/or R162A. Examples of the above-referenced modifications are described in co-pending U.S. Patent Publication Nos. 2003/0104988, 2002/0086816 and 2003/0072737, which are incorporated by reference herein in their entirety. In the mutein nomenclature used herein, the changed amino acid is depicted with the native amino acid's one letter code first, followed by its position in the EPO molecule, followed by the replacement amino acid one letter code. For example, S100E refers to a human EPO molecule in which, at amino acid 100, the serine has been changed to a glutamic acid.

In another embodiment, the tissue protective cytokine may include one or more of the above site mutations, providing that the site mutations do not include I6A, C7A, K20A, P42A, D43A, K45D, K45A, F48A, Y49A, K52A, K49A, S100B, R103A, K116A, T132A, I133A, K140A, N147K, N147A, R150A, R150E, G151A, K152A, K154A, G158A, C161A, or R162A.

In still another embodiment, the tissue protective cytokines may include combinations of site mutations, such as K45D/S100E, K97D/S100E, A30N/H32T, K45D/R150E, R103E/L108S, K140A/K52A, K140A/K52A/K45A, K97A/K152A, K97A/K152A/K45A, K97A/K152A/K45A/K52A, K97A/K152A/K45A/K52A/K140A, K97A/K152A/K45A/K52A/K140A/K154A, N24K/N38K/N83K, and N24K/Y15A. In yet another embodiment, the tissue protective cytokines do not include any of the above combinations. In another embodiment, the tissue protective cytokines may include any of the above-referenced site mutations providing that the site mutations do not include any of the following combinations of substitutions: N24K/N38K/N83K and/or A30N/H32T.

Certain modifications or combinations of modifications may affect the flexibility of the mutein's ability to bind with its receptor, such as an EPO receptor or secondary receptor. Examples of such modifications or combinations of modifications include, but are not limited to, K152W, R14A/Y15A, I6A, C7A, D43A, P42A, F48A, Y49A, T132A, I133A, T134A, N147A, P148A, R150A, G151A, G158A, C161A, and R162A. Corresponding mutations are known to those of ordinary skill in the art to be detrimental in human growth hornone. Thus, in one embodiment, the tissue protective cytokine does not include one or more of the modifications or combinations of modifications that may affect the flexibility of the mutein's ability to bind with its receptor. Further discussion of such tissue protective cytokines is included in co-pending U.S. patent application Ser. No. 10/612,665, attorney docket no. 10165-022-999, filed Jul. 1, 2003, entitled “Recombinant Tissue Protective Cytokines and Encoding Nucleic Acids Thereof for Protection, Restoration, and Enhancement of Responsive Cells, Tissues, and Organs,” the entire disclosure of which is incorporated by reference herein

Moreover, suitable tissue protective cytokines for use with the present invention includes the long acting chemically modified EPO molecules disclosed in International Application No. US03/028073, filed under attorney docket no. 20528.0006 on Sep. 9, 2003, entitled “Long Acting Erythropoietins that Maintain Tissue Protective Activity of Endogeneous Erythropoietin”, which is incorporated in its entirety by reference herein. For example, suitable tissue protective cytokines for use with the present invention includes EPO that has undergone at least one chemical modification to at least one of the N-linked oligosaccharide chains or the O-linked oligosaccharide chain, wherein the chemical modification includes oxidation, sulfation, phosphorylation, PEGylation, or a combination thereof. In one embodiment, the EPO molecule subject to chemical modification is in α form. In another embodiment, the EPO molecule subject to chemical modification is in β form. In yet another embodiment, the EPO molecule subject to chemical modification is asialic. In yet another embodiment, the EPO molecule subject to chemical modification may be ARANESP (Amgen, Thousand Oaks, Calif.) or CERA (Hoffmann-La Roche Inc., Nutley, N.J.).

Recombinant Techniques

A variety of host-expression vector systems may be utilized to produce the compounds. Such host-expression systems represent vehicles by which the compound of interest may be produced and subsequently purified, but also represent cells that may, when transformed or transfected with the appropriate nucleotide coding sequences, exhibit the modified erythropoietin gene product in situ. These include but are not limited to, bacteria, insect, plant, mammalian, including human host systems, such as, but not limited to, insect cell systems infected with recombinant virus expression vectors (e.g., baculovirus) containing the compound coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing erythropoietin-related molecule coding sequences; or mammalian cell systems, including human cell systems, e.g., HT1080, COS, CHO, BHK, 293, 3T3, harboring recombinant expression constructs containing promoters derived from the genome of mammalian cells, e.g., metallothionein promoter, or from mammalian viruses, e.g., the adenovirus late promoter; the vaccinia virus 7.5K promoter.

In addition, a host cell strain may be chosen that modulates the expression of the inserted sequences, or modifies and processes the gene product in the specific fashion desired. Such modifications and processing of protein products may be important for the function of the protein. As known to those of ordinary skill in the art, different host cells have specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells, including human host cells, include but are not limited to HT1080, CHO, VERO, BHK, HeLa, COS, MDCK, 293, 3T3, and WI38.

For long-term, high-yield production of recombinant compounds, stable expression is preferred. For example, cell lines that stably express the recombinant tissue protective cytokine-related molecule gene product may be engineered. Rather than using expression vectors that contain viral origins of replication, host cells can be transformed with DNA controlled by appropriate expression control elements, e.g., promoter, enhancer, sequences, transcription terminators, polyadenylation sites, and the like, and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express the tissue-protective product. Such engineered cell lines may be particularly useful in screening and evaluation of compounds that affect the endogenous activity of the EPO-related molecule gene product.

### Purification of Compounds

The compounds of the invention can be purified by art-known techniques such as reverse phase chromatography high performance liquid chromatography, ion exchange chromatography, gel electrophoresis, affinity chromatography and the like. The actual conditions used to purify a particular peptide will depend, in part, on synthesis strategy and on factors such as net charge, hydrophobicity, hydrophilicity, etc., and will be apparent to those having skill in the art. Compounds can be purified, e.g., by ion exchange or size exclusion chromatography.

For affinity chromatography purification, any antibody which specifically binds the compound may be used. For the production of antibodies, various host animals, including but not limited to rabbits, mice, rats, etc., may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a compound may be prepared using any technique which provides for the production of antibody molecules by continuous cell lines in culture. These include but are not limited to the hybridoma technique originally described by Kohler and Milstein, 1975, Nature 256:495-497, or Kaprowski, U.S. Pat. No. 4,376,110; the human B-cell hybridoma technique (Kosbor et al., 1983, Immunology Today 4:72; Cote et al., 1983, Proc. Natl. Acad. Sci. USA 80:2026-2030); and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. USA 81:6851-6855; Neuberger et al., 1984, Nature 312:604-608; Takeda et al., 1985, Nature 314:452-454, Boss, U.S. Pat. No. 4,816,397; Cabilly, U.S. Pat. No. 4,816,567) by splicing the genes from a mouse antibody molecule of appropriate antigen specificity together with genes from a human antibody molecule of appropriate biological activity can be used. Or “humanized” antibodies can be prepared (see, e.g., Queen, U.S. Pat. No. 5,585,089). Alternatively, techniques described for the production of single chain antibodies (U.S. Pat. No. 4,946,778) can be adapted to produce peptide-specific single chain antibodies. Antibody fragments which contain deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the compound of interest.

The antibody or antibody fragment specific for the desired compound can be attached, for example, to agarose, and the antibody-agarose complex is used in immunochromatography to purify compounds of the invention. See, Scopes, 1984, Protein Purification: Principles and Practice, Springer-Verlag New York, Inc., N.Y., Livingstone, 1974, Methods In Enzymology: Immunoaffinity Chromatography of Proteins 34:723-731.

Further Modifications

Compounds with additional modifications can also be used in the method of the present invention for preventing, treating, ameliorating or managing cancer, neoplastic disease, the symptoms thereof, or the side effects associated with other treatments thereof, such as chemotherapy or radiation. For example, the compounds may be made to incorporate one or more (D)-amino acids in some embodiments. The choice of including an (L)- or (D)-amino acid into a compound of the present invention depends, in part, upon the desired characteristics of the compound. For example, the incorporation of one or more (D)-amino acids can confer increasing stability on the compound in vitro or in vivo. The incorporation of one or more (D)-amino acids can also increase or decrease the binding activity of the compound as determined, for example, using the bioassays described herein, or other methods well known in the art.

Amino acid “modification” refers to the alteration of a naturally occurring amino acid to produce a non-naturally occurring amino acid. Derivatives of the compounds of the present invention with non-naturally occurring amino acids can be created by chemical synthesis or by site specific incorporation of unnatural amino acids into peptides during biosynthesis, as described in Christopher J. Noren, Spencer J. Anthony-Cahill, Michael C. Griffith, Peter G Schultz, 1989 Science, 244:182-188, hereby incorporated by reference herein in its entirety.

Mimetics that are structurally similar to therapeutically useful compounds may be used to produce an equivalent therapeutic or prophylactic effect. For example, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂—NH—, —CH₂S—, —CH₂—CH₂—, —CH═CH-(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p 267 (1983); Spatola, A. F., Vega Data (March 1983), Vol. 1. Issue 3, “Peptide Backbone Modifications” (general review); Morely, J. S., Trends Pharma Sci (1980) pp. 463-468 (general review); Hudson, D. et al., (1979) Int J Pept Prot Re 14: 177-185 (—CH₂—NH—, —CH₂—CH₂—); Spatola, A. F. et al., (1986) Life Sci 38:1243-1249 (—CH₂—S); Hann, M. M., (1982) J Chem Soc Perkin Trans 1307-314 (—CH═CH—, cis and trans); Almquist, R. G et al., (1980) J Med Chem 23: 1392 (—COCH₂—); Jennings-White, C et al., (1982) Tetrahedron Lett 23:2533 (—COCH₂—); Szelke, Metal., European Appln. EP 45665 (1982) CA: 97: 39405 (1982) (—CH(OH)CH₂—); Holladay, M. W. et al., (1983) Tetrahedron Lett 24:4401-4404 (—C(OH)CH₂—); and Hruby, V. J., (1982) Life Sci 31:189-199 (—CH₂—S—); each of which is incorporated herein by reference.

In another embodiment, a particularly preferred non-peptide linkage is —CH₂NH—. Such mimetics may have significant advantages over compound embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

A variety of designs for mimetics are possible. For example, cyclic peptides, in which the necessary conformation is stabilized by non-peptides, are specifically contemplated, U.S. Pat. No. 5,192,746 to Lobl, et al., U.S. Pat. No. 5,576,423 to Aversa, et al., U.S. Pat. No. 5,051,448 to Shashoua, and U.S. Pat. No. 5,559,103 to Gaeta, et al., all hereby incorporated by reference, describe multiple methods for creating such compounds. Synthesis of nonpeptide compounds that mimic peptide sequences is also known in the art. Eldred et al., J. Med. Chem. 37:3882 (1994), hereby incorporated by reference herein in its entirety) describe non-peptide antagonists that mimic the peptide sequence. Likewise, Ku et al., J. Med. Chem 38:9 (1995) (hereby incorporated by reference herein in its entirety) further elucidates the synthesis of a series of such compounds.

Further modifications following synthesis may be implemented. For example, the compounds may be further chemically modified, i.e. carbamoylated, acetylated, succinylated, guanidated, nitrated, trinitrophenylated, amidinated, etc., in accordance with U.S. patent application Ser. No. 10/188,905, which published as 20030072737-A1 on Apr. 17, 2003 and discloses chemically modified EPO, and in accordance with U.S. patent application Ser. No. 10/612,665, filed Jul. 1, 2003, and U.S. patent application Ser. No. 09/753,132, filed Dec. 29, 2000, which are incorporated by reference herein in their entirety.

Additionally, the compounds may consist of recombinant compounds—muteins. The disclosed mutations may include substitutions, deletions, including internal deletions, additions, including additions yielding fusion proteins, or conservative substitutions of amino acid residues within and/or adjacent to the amino acid sequence, but that result in a “silent” change, and non-conservative amino acid changes and larger insertions and deletions, as previously disclosed in PCT/US03/20964 entitled Recombinant Tissue Protective Cytokines and Encoding Nucleic Acids Thereof for Protection, Restoration, and Enhancement of Responsive Cells, Tissues, and Organs (which is incorporated by reference herein in its entirety)

Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar (hydrophobic)=cysteine, alanine, valine, leucine, isoleucine, proline, phenylalanine, methionine, tryptophan, glycine, tyrosine; and (4) uncharged polar=asparagine, glutamine, serine, threonine. Non-polar may be subdivided into: strongly hydrophobic=alanine, valine, leucine, isoleucine, methionine, phenylalanine and moderately hydrophobic=glycine, proline, cysteine, tyrosine, tryptophan. In alternative fashion, the amino acid repertoire can be grouped as (1) acidic=aspartate, glutamate; (2) basic=lysine, arginine, histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, WH Freeman and Co., 1995, which is incorporated by reference herein in its entirety).

Alternatively, mutations can be introduced randomly along all or part of the coding sequence of a compound, such as by saturation mutagenesis, and the resultant mutants can be screened for biological activity to identify mutants that retain activity. Following mutagenesis, the encoded peptide can be expressed recombinantly and the activity of the recombinant peptide can be determined.

In another embodiment, the compound may be further modified through the additions of polymers (such as polyethylene glycol), sugars, or additional proteins (such as a fusion construct) in an effort to extend the half-life of the peptide or enhance the peptide's tissue protective effects. Examples of such modifications are disclosed within WO/04022577 A3 and WO/05025606 A1, which are incorporated herein by reference.

In one embodiment, the PEG is an amino PEG, preferably a methoxy PEG with primary amino groups at the termini (mPEG-NH₂). Polyethylene glycol chains with primary amino groups at the termini are very useful functionalized polymers. The amino end groups on mPEG-NH₂ are more reactive toward acylating agents than the hydroxyl groups that are present on conventional PEGs and they also readily undergo reductive amination reactions. In another embodiment, the PEG is an electrophilically activated PEG, such as mPEG-succinimidyl propionate (mPEG-SPA) or mPEG-succinimidyl butanoate (mPEG-SBA), both of which are commercially available from Nektar Therapeutics of Birmingham, Ala. In yet another embodiment, the PEG is a methoxy PEG-hydrazide.

In one embodiment, the addition of the at least one PEG is achieved via oxidation with periodate (as disclosed above), followed by the use of cyanoborohydride and an amino PEG. For example, EPO in solution may be first oxidized with a periodate, e.g., sodium periodate, for a predetermined period of time at room temperature, which produces aldehydes in the carbohydrate chains. A suitable periodate is sodium meta-periodate, which is commercially available from Sigma. The periodate may then be removed by buffer exchange, at which time the oxidized sialic acid groups on N-linked oligosaccharide groups of EPO may be subjected to at least one amino PEG in the presence of cyanoborohydride. Suitable PEGs for use include, but are not limited to, methoxy-PEG-hydrazides, which are commercially available from Nektar Therapeutics.

In another embodiment, the addition of the at least one PEG is performed by the attachment of PEG groups to terminal galactose residues after oxidation with galactose oxidase. For example, an asialo form of EPO (having exposed terminal galactose residues) in buffer is first subjected to galactose oxidase (commercially available from Sigma) to generate aldehydes in the carbohydrate chains. The buffer may then be removed by buffer exchange, at which time the oxidized galactose residues may be subjected to at least one amino PEG in the presence of cyanoborohydride.

Further, the use of PEG to derivatize peptide therapeutics has been demonstrated to reduce the immunogenicity of the peptides. For example, U.S. Pat. No. 4,179,337 (Davis et al.) discloses non-immunogenic polypeptides such as enzymes and peptide hormones coupled to polyethylene glycol (PEG) or polypropylene glycol. In addition to reduced immunogenicity, the clearance time in circulation is prolonged due to the increased size of the PEG-conjugate of the polypeptides in question.

The principal mode of attachment of PEG, and its derivatives, to peptides proteins is a non-specific bonding through a peptide amino acid residue (see e.g., U.S. Pat. No. 4,088,538 U.S. Pat. No. 4,496,689, U.S. Pat. No. 4,414,147, U.S. Pat. No. 4,055,635, and PCT WO 87/00056). Another mode of attaching PEG to peptides is through the non-specific oxidation of glycosyl residues on a glycopeptide (see e.g., WO 94/05332). In these non-specific methods, poly(ethyleneglycol) is added in a random, non-specific manner to reactive residues on a peptide backbone.

Specifically labeled, homogeneous peptide therapeutics can be produced in vitro through the action of enzymes. Unlike the typical non-specific methods for attaching a synthetic polymer or other label to a peptide, enzyme-based syntheses have the advantages of regioselectivity and stereo selectivity. Two principal classes of enzymes for use in the synthesis of labeled peptides are glycosyltransferases (e.g., sialyltransferases, oligosaccharyltransferases, N-acetylglucosaminyltransferases), and glycosidases. These enzymes can be used for the specific attachment of sugars which can be subsequently modified to comprise a therapeutic moiety. Alternatively, glycosyltransferases and modified glycosidases can be used to directly transfer modified sugars to a peptide backbone (see e.g., U.S. Pat. No. 6,399,336, and U.S. Patent Application Publications 20030040037, 20040132640, 20040137557, 20040126838, and 20040142856, each of which are incorporated by reference herein). Methods combining both chemical and enzymatic synthetic elements are also known (see e.g., Yamamoto et al. Carbohydr. Res. 305: 415-422 (1998) and U.S. Patent Application Publication 20040137557 which is incorporated herein by reference).

### 7. AS SAYS FOR TESTING COMPOUNDS

7.1 Inhibition of Cancer and Neoplastic Cells and Disease

The isolated compounds to be used within the method of the current invention may be demonstrated to inhibit tumor cell proliferation, cell transformation and tumorigenesis in vitro or in vivo using a variety of assays known in the art, or described herein. Such assays can use cells of a cancer cell line or cells from a patient. Many assays well-known in the art can be used to assess such survival and/or growth; for example, cell proliferation can be assayed by measuring (³H)-thymidine incorporation, by direct cell count, by detecting changes in transcription, translation or activity of known genes such as proto-oncogenes (e.g., fos, myc) or cell cycle markers (Rb, cdc2, cyclin A, D1, D2, D3 or E). The levels of such protein and mRNA and activity can be determined by any method well known in the art. For example, protein can be quantitated by known immunodiagnostic methods such as Western blotting or immunoprecipitation using commercially available antibodies (for example, many cell cycle marker antibodies are from Santa Cruz, Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art. Differentiation can be assessed visually based on changes in morphology, etc.

The present invention provides for cell cycle and cell proliferation analysis by a variety of techniques known in the art, including but not limited to the following:

As one example, bromodeoxyuridine (“BRDU”) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38, 369; Campana et al., 1988, J. Immunol. Meth. 107, 79).

Cell proliferation may also be examined using (3H)-thymidine incorporation (see e.g., Chen, J., 1996, Oncogene 13:1395 403; Jeoung, J., 1995, J. Biol. Chem. 270:18367 73). This assay allows for quantitative characterization of S-phase DNA synthesis. In this assay, cells synthesizing DNA will incorporate (³H)-thyrnidine into newly synthesized DNA. Incorporation may then be measured by standard techniques in the art such as by counting of radioisotope in a Scintillation counter (e.g., Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189 199; Vassilev et al., 1995, J. Cell Sci. 108:1205 15).

Cell proliferation may be measured by counting samples of a cell population over time (e.g., daily cell counts). Cells may be counted using a hemacytometer and light microscopy (e.g., HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g., cells in S-phase) will exhibit a ploidy value higher than 2N and up to 4N DNA content. Ploidy value and cell-cycle kinetics may be further measured using propidum iodide assay (see e.g., Turner, T., et al., 1998, Prostate 34:175 81). Alternatively, the DNA ploidy may be determined by quantitation of DNA Feulgen staining (which binds to DNA in a stoichiometric manner) on a computerized microdensitometrystaining system (see e.g., Bacus, S., 1989, Am. J. Pathol. 135:783 92). In another embodiment, DNA content may be analyzed by preparation of a chromosomal spread (Zabalou, S., 1994, Hereditas. 120:127 40; Pardue, 1994, Meth. Cell Biol. 44:333 351).

The expression of cell-cycle proteins (e.g., CycA. CycB, CycE, CycD, cdc2, Cdk4/6, Rb, p21 or p2′7) provide crucial information relating to the proliferative state of a cell or population of cells. For example, identification in an anti-proliferation signaling pathway may be indicated by the induction of p21cip1. Increased levels of p21 expression in cells results in delayed entry into G1 of the cell cycle (Harper et al., 1993, Cell 75:805 816; Li et al., 1996, Curr. Biol. 6:189 199). p21 induction may be identified by immunostaining using a specific anti-p21 antibody available commercially (e.g., from Santa Cruz, Inc.). Similarly, cell-cycle proteins may be examined by Western blot analysis using commercially available antibodies. In another embodiment, cell populations are synchronized prior to detection of a cell cycle protein. Cell-cycle proteins may also be detected by FACS (fluorescence-activated cell sorter) analysis using antibodies against the protein of interest.

Detection of changes in length of the cell cycle or speed of cell cycle may also be used to measure inhibition of cell proliferation by a Compound of the Invention. In one embodiment the length of the cell cycle is determined by the doubling time of a population of cells (e.g., using cells contacted or not contacted with one or more Compounds of the Invention). In another embodiment, FACS analysis is used to analyze the phase of cell cycle progression, or purify G1, S, and G2/M fractions (see e.g., Delia, D. et al., 1997, Oncogene 14:2137 47).

Lapse of cell cycle checkpoint(s), and/or induction of cell cycle checkpoint(s), may be examined by the methods described herein, or by any method known in the art. Without limitation, a cell cycle checkpoint is a mechanism which ensures that a certain cellular events occur in a particular order. Checkpoint genes are defined by mutations that allow late events to occur without prior completion of an early event (Weinert, T., and Hartwell, L., 1993, Genetics, 134:63 80). Induction or inhibition of cell cycle checkpoint genes may be assayed, for example, by Western blot analysis, or by immunostaining, etc. Lapse of cell cycle checkpoints may be further assessed by the progression of a cell through the checkpoint without prior occurrence of specific events (e.g. progression into mitosis without complete replication of the genomic DNA).

In addition to the effects of expression of a particular cell cycle protein, activity and post-translational modifications of proteins involved in the cell cycle can play an integral role in the regulation and proliferative state of a cell. The invention provides for assays involved detected post-translational modifications (e.g., phosphorylation) by any method known in the art. For example, antibodies that detect phosphorylated tyrosine residues are commercially available, and may be used in Western blot analysis to detect proteins with such modifications. In another example, modifications such as myristylation, may be detected on thin layer chromatography or reverse phase h.p.l.c. (see e.g., Glover, C., 1988, Biochem. J. 250:485 91; Paige, L., 1988, Biochem J.; 250:485 91).

Activity of signaling and cell cycle proteins and/or protein complexes is often mediated by a kinase activity. The present invention provides for analysis of kinase activity by assays such as the histone H1 assay (see e.g., Delia, D. et al., 1997, Oncogene 14:2137 47).

The Compounds used within the method of the Invention can also be demonstrated to alter cell proliferation in cultured cells in vitro using methods which are well known in the art. Specific examples of cell culture models include, but are not limited to, for lung cancer, primary rat lung tumor cells (Swafford et al., 1997, Mol. Cell. Biol., 17:1366 1374) and large-cell undifferentiated cancer cell lines (Mabry et al., 1991, Cancer Cells, 3:53 58); colorectal cell lines for colon cancer (Park and Gazdar, 1996, J. Cell Biochem. Suppl. 24:131 141); multiple established cell lines for breast cancer (Hambly et al., 1997, Breast Cancer Res. Treat. 43:247 258; Gierthy et al., 1997, Chemosphere 34:1495 1505; Prasad and Church, 1997, Biochem. Biophys. Res. Commun. 232:14 19); a number of well-characterized cell models for prostate cancer (Webber et al., 1996, Prostate, Part 1, 29:386 394; Part 2, 30:58 64; and Part 3, 30:136 142; Boulikas, 1997, Anticancer Res. 17:1471 1505); for genitourinary cancers, continuous human bladder cancer cell lines (Ribeiro et al., 1997, Int. J. Radiat. Biol. 72:11 20); organ cultures of transitional cell carcinomas (Booth et al., 1997, Lab Invest. 76:843 857) and rat progression models (Vet et al., 1997, Biochim. Biophys Acta 1360:39 44); and established cell lines for leukemias and lymphomas (Drexler, 1994, Leuk. Res. 18:919 927, Tohyama, 1997, Int. J. Hematol. 65:309 317).

The Compounds used in the method of treatment can also be demonstrated to inhibit cell transformation (or progression to malignant phenotype) in vitro. In this embodiment, cells with a transformed cell phenotype are contacted with one or more compounds, and examined for change in characteristics associated with a transformed phenotype (a set of in vitro characteristics associated with a tumorigenic ability in vivo), for example, but not limited to, colony formation in soft agar, a more rounded cell morphology, looser substratum attachment, loss of contact inhibition, loss of anchorage dependence, release of proteases such as plasminogen activator, increased sugar transport, decreased serum requirement, or expression of fetal antigens, etc. (see Luria et al., 1978, General Virology, 3d Ed., John Wiley & Sons, New York, pp. 436 446).

Loss of invasiveness or decreased adhesion may also be used to demonstrate the anti-cancer effects of the compounds used in the method of the Invention. For example, a critical aspect of the formation of a metastatic cancer is the ability of a precancerous or cancerous cell to detach from primary site of disease and establish a novel colony of growth at a secondary site. The ability of a cell to invade peripheral sites is reflective of a potential for a cancerous state. Loss of invasiveness may be measured by a variety of techniques known in the art including, for example, induction of E-cadherin-mediated cell-cell adhesion. Such E-cadherin-mediated adhesion can result in phenotypic reversion and loss of invasiveness (Hordijk et al., 1997, Science 278:1464 66).

Loss of invasiveness may further be examined by inhibition of cell migration. A variety of 2-dimensional and 3-dimensional cellular matrices are commercially available (Calbiochem-Novabiochem Corp. San Diego, Calif.). Cell migration across or into a matrix may be examined by microscopy, time-lapsed photography or videography, or by any method in the art allowing measurement of cellular migration. In a related embodiment, loss of invasiveness is examined by response to hepatocyte growth factor (HGF). HGF-induced cell scattering is correlated with invasiveness of cells such as Madin-Darby canine kidney (MDCK) cells. This assay identifies a cell population that has lost cell scattering activity in response to HGF (Hordijk et al., 1997, Science 278:1464 66).

Alternatively, loss of invasiveness may be measured by cell migration through a chemotaxis chamber (Neuroprobe/Precision Biochemicals Inc. Vancouver, BC). In such assay, a chemo-attractant agent is incubated on one side of the chamber (e.g., the bottom chamber) and cells are plated on a filter separating the opposite side (e.g., the top chamber). In order for cells to pass from the top chamber to the bottom chamber, the cells must actively migrate through small pores in the filter. Checkerboard analysis of the number of cells that have migrated may then be correlated with invasiveness (see e.g., Ohnishi, T., 1993, Biochem. Biophys. Res. Commun. 193:518 25).

The compounds used in the method of the Invention can also be demonstrated to inhibit tumor formation in vivo. A vast number of animal models of hyperproliferative disorders, including tumorigenesis and metastatic spread, are known in the art (see Table 317-1, Chapter 317, “Principals of Neoplasia,” in Harrison's Principals of Internal Medicine, 13th Edition, Isselbacher et al., eds., McGraw-Hill, N.Y., p. 1814, and Lovejoy et al., 1997, J. Pathol. 181:130 135). Specific examples include for lung cancer, transplantation of tumor nodules into rats (Wang et al., 1997, Ann. Thorac. Surg. 64:216 219) or establishment of lung cancer metastases in SCID mice depleted of NK cells (Yono and Sone, 1997, Gan To Kagaku Ryoho 24:489 494); for colon cancer, colon cancer transplantation of human colon cancer cells into nude mice (Gutman and Fidler, 1995, World J. Surg. 19:226 234), the cotton top tamarin model of human ulcerative colitis (Warren, 1996, Aliment. Pharmacol. Ther. 10 Supp 12:45 47) and mouse models with mutations of the adenomatous polyposis tumor suppressor (Polakis, 1997, Biochim. Biophys. Acta 1332:F127 F147); for breast cancer, transgenic models of breast cancer (Dankort and Muller, 1996, Cancer Treat. Res. 83:71 88; Amundadittir et al., 1996, Breast Cancer Res. Treat. 39:119 135) and chemical induction of tumors in rats (Russo and Russo, 1996, Breast Cancer Res. Treat. 39:7 20); for prostate cancer, chemically-induced and transgenic rodent models, and human xenograft models (Royai et al., 1996, Semin. Oncol. 23:35 40); for genitourinary cancers, induced bladder neoplasm in rats and mice (Oyasu, 1995, Food Chem. Toxicol 33:747 755) and xenografts of human transitional cell carcinomas into nude rats (Jarrett et al., 1995, J. Endourol. 9:1 7); and for hematopoietic cancers, transplanted allogeneic marrow in animals (Appelbaum, 1997, Leukemia 11 (Suppl. 4): S15 S17). Further, general animal models applicable to many types of cancer have been described, including, but not restricted to, the p53-deficient mouse model (Donehower, 1996, Semin. Cancer Biol. 7:269 278), the Min mouse (Shoemaker et al., 1997, Biochem. Biophys. Acta, 1332:F25 F48), and immune responses to tumors in rat (Frey, 1997, Methods, 12:173 188). Additionally, for example, a compounds ability to arrest or retard the growth of a tumor in vivo may be verified using a 9L rat gliasarcoma model as disclosed in (Murphy et al. 2007, J Neurooncol. 85:181-189). In this model, 9L gliasarcoma are implanted into rats. The 9L gliasarcoma may be derived from a piece of tumor that is implanted or from a cell culture. Further, the 9L gliasarcoma may be implanted subcutaneously as disclosed in Murphy et al.

For example, a compound to be used in the method of the Invention can be administered to a test animal, in one embodiment a test animal predisposed to develop a type of tumor, and the test animal subsequently examined for a decreased incidence of tumor formation in comparison with an animal not administered the compound. Alternatively, a compound can be administered to test animals having tumors (e.g., animals in which tumors have been induced by introduction of malignant, neoplastic, or transformed cells, or by administration of a carcinogen) and subsequently examining the tumors in the test animals for tumor regression in comparison to animals not administered the compound.

Further compounds used within the method of the Invention, may be tested in various in vivo assays in the art to determine their ability to prevent, treat, ameliorate, or manage the symptoms associated with cancer. For example, the compound's ability to address cachexia may be evaluated in vitro using an interleukin-6 assay disclosed in Kuroda et al., Clinical Cancer Research 2005 11:5590-5594. Additionally, in vivo assays, including, but not limited to, the Yoshida AH-130 rat ascites hepatoma assay (Carbó et al., British Journal of Cancer (2000) 83(4):526-531; Costelli et al., Am J Physiol Regul Integr Comp Physiol (2006) 291:R674-R683); T-Cell Targeted Human Tumor Necrosis Factor murine model (Probert et al., (1993) 151(4): 1894-1906); R-1 clone murine model (Lazarus et al. Am J Physiol Endocrinol Metab (1999) 277:E332-E341); and human prostate cancer (JCA-1) murine model (Kuroda et al., Clinical Cancer Research 2005 11:5590-5594) are useful in evaluating the compounds ability to prevent, treat, manage or ameliorate cachexia associated with cancer or neoplastic diseases.

Further a compounds ability to prevent, treat, ameliorate or manage the various syndromes associated with cancer can be evaluated using well known models in the art. For example, several animal models have been generated such as the ApcMin Mouse, 1638n, and ApcPirc models for Familial adenomatous polyposis (Amos-Landgraf J, Kwong L N, Dove W F, et al (2007). “A target-selected Apc-mutant rat kindred enhances the modeling of familial human colon cancer.”. PNAS 104 (10): 4036-4041); 1638N: A mouse model for familial adenomatous polyposis-associated desmoid tumors and cutaneous cysts. Gastroenterology, Volume 114, Issue 2, Pages 275-283 R. Smits, W. van der Houven van Oordt, A. Luz, C. Zurcher, S. Jagmohan-Changur, C. Breukel, P. Khan, R. Fodde; Hiai H, Hino O (eds): Animal Models of Cancer Predisposition Syndromes Prog Exp Tumor Res. Basel, Karger, 1999, vol 35, pp 109-119 (DOI: 10.1159/000062007); knock out and transgenic mice models as well as an animal model involving immunization with the acetylcholine receptor as models of myasthenia gravis (Erdem Tüzün Unraveling myasthenia gravis immunopathogenesis using animal models Drug Discovery Today: Disease Models Volume 3, Issue 1, Spring 2006, Pages 15-20); Fhit-deficient mice as a model of Muir-Tone syndrome (Fong et al., Muir-Tone-like syndrome in Fhit-deficient mice. Proc. Nat. Acad. Sci. 97: 4742-4747, 2000.)

7.2 Tissue Protective Assays and Models

Preferably, the compounds utilized in the current method also exhibit tissue protective properties as well, i.e. anti-apoptoitc, neuritogenic, anti-inflamatory, neuroprotective, etc. Compounds in accordance with the present invention may be tested for tissue protective activity, e.g., protecting cells, tissues or organs. Protective activities may be further tested using in vitro and in vivo assays. In vitro tests that are indicative of tissue protective activity include, for example, cell proliferation assays, cell differentiation assays, or detecting the presence of proteins or nucleic acids upregulated by tissue protective receptor complex, e.g. tissue protective cytokine receptor complex, activity, e.g., nucleolin, neuroglobin, cytoglobin, or frataxin. Neuroglobin, for example, may be involved in facilitating the transport or the short-term storage of oxygen. Therefore, oxygen transport or storage assays may be used as an assay to identify or screen for compounds which modulate tissue protective activity.

Neuroglobin is expressed in cells and tissues of the central nervous system in response to hypoxia or ischemia and may provide protection from injury (Sun et al. 2001, PNAS 98:15306-15311; Schmid et al., 2003, J. Biol. Chem. 276:1932-1935, each of which is incorporated by reference herein in its entirety). Cytoglobin may play a similar role in protection, but is expressed in a variety of tissues at varying levels (Pesce et al., 2002, EMBO 3:1146-1151, which is incorporated by reference herein in its entirety). In one embodiment of the invention, the levels of an upregulated protein in a cell may be measured before and after contacting the compound to a cell. In certain embodiments, the presence of an upregulated protein associated with tissue protective activity in a cell, may be used to confirm the tissue protective activities of a compound.

Nucleolin may protect cells from damage. It plays numerous roles in cells including modulation of transcription processes, sequence specific RNA-binding protein, cytokinesis, nucleogensis, signal transduction, apoptosis induced by T-cells, chromatin remodelling, or replication. It can also function as a cell surface receptor DNA/RNA helicase, DNA-dependent ATPase, protein shuttle, transcription factor component, or transcriptional repressor (Srivastava and Pollard, 1999, FASEB J., 13:1911-1922; and Ginisty et al., 1999, J. Cell Sci., 112:761-772, each of which is incorporated by reference herein in its entirety).

Frataxin is a protein involved with mitochondrial iron metabolism and has previously been shown to be strongly up-regulated by EPO both in vivo and in vitro (Sturm et al. (2005) Eur J Clin Invest 35: 711, which is incorporated by reference herein in its entirety)

Expression of an upregulated protein may be detected by detecting mRNA levels corresponding to the protein in a cell. The mRNA can be hybridized to a probe that specifically binds a nucleic acid encoding the upregulated protein. Hybridization may consist of, for example, Northern blot, Southern blot, array hybridization, affinity chromatography, or in situ hybridization.

Tissue protective activity of the compound of the invention can also be detected using in vitro neuroprotection assays. For example, primary neuronal cultures may be prepared from new born rat hippocampi by trypsinization, and cultured as by any method known in the art and/or described herein e.g. in MEM-II growth medium (Invitrogen), 20 mM D-glucose, 2 mM L-glutamine, 10% Nu-serum (bovine; Becton Dickinson, Franklin Lakes, N.J.), 2% B27 supplement (Invitrogen), 26.2 mM NaHCO3, 100 U/ml penicillin, and 1 mg/ml streptavidin (see, e.g., Leist et al., 2004, Science 305:239-242, hereby incorporated by reference in its entirety). One day after seeding, 1 μM cytosinearabino-furanoside is added. Thirteen day old cultures are then preincubated with increasing doses of EPO or CEPO (3-3000 pM) for 24 h. On day 14, the medium is removed and the cultures challenged with 300 μM NMDA in PBS at RT. After 5 min, pre-conditioned medium is returned to the cultures which are then returned to the incubator for 24 h. The cells are fixed in paraformaldehyde, stained by Hoechst 33342 (Molecular Probes, Eugene, Oreg.) and condensed apoptotic nuclei may be counted. NGF (50 ng/ml) and MK801 (1 μM) are included as positive controls.

Animal model systems can be used to demonstrate the tissue protective activity of a compound or to demonstrate the safety and efficacy of the compounds identified by the screening methods of the invention described above. The compounds identified in the assays can then be tested for biological activity using animal models for a type of tissue damage, disease, condition, or syndrome of interest. These include animals engineered to contain the tissue protective receptor complex coupled to a functional readout system, such as a transgenic mouse.

Animal models that can be used to test the efficacy of the cell or tissue protective activity of an identified compound are known in the art and include, for example, protection against the onset of acute experimental allergic encephalomyelitis in Lewis rats, restoration or protection from diminished cognitive function in mice after receiving brain trauma, cerebral ischemia (“stroke”) or seizures stimulated by excitotoxins (Brines et al., 2000, PNAS, 97:10295-10672, which is incorporated by reference herein in its entirety), protection from induced retinal ischemia (Rosenbaum et al., 1997, Vis. Res. 37:3443-51 which is incorporated by reference herein in its entirety), protection from injury to the sciatic nerve, and protection from ischemia-reperfusion injury to the heart (in vitro cardiomyocyte studies and in vivo ischemia-reperfusion injury, see, e.g., Calvillo et al., 2003, PNAS 100:4802-4806 and Fiordaliso et al., 2005, PNAS 102:2046-2051, each of which is hereby incorporated by reference in its entirety). Such assays are described in further detail in Grasso et al. (2004) Med Sci Monit 10: BR1-3, PCT publication no. WO02/053580, or PCT application PCT/US2006/031061 each of which is incorporated by reference herein in its entirety. The in vivo methods described therein are directed towards administration of EPO, however, tissue protective proteins administered in place of EPO have been identified to also exhibit similar biologic activity, e.g., Leist et al. (2004) Science 305: 239-242, which is incorporated by reference herein in its entirety. Compounds may be substituted for testing as well. Other assays for determining tissue protective activity of a compound are well known to those of skill in the art.

Alternatively, cell binding assays can be for evaluation of the compounds of the invention. For example, the compound of interest can be bound to a biological marker such as a fluorescent or radiolabled marker for ease of detection and tested for binding to transfected BaF3 cells expressing EPOR and/or β_{c} receptor. In a 96 well plate, eight 1:2 serial dilutions of the compound of interest in growth medium (RPMI 1640, 10% fetal bovine serum, 1 mM sodium pyruvate, 2 mM L-glutamine) are plated, such that the final volume in each well is about 100 μl. The BaF3 parental line and BaF3 cells transfected with EPOR and/or β_{c} receptor can be washed three times in growth media (see above), pellets resuspended in growth medium, and cells counted and diluted in growth media to 5,000 cells/100 μl. 100 μl of diluted cells are then added to each compound dilution. The assay plate is then incubated in a 37° C. incubator for three to four days. The plate/cells are then washed and the plate is read on a fluorescent plate reader or by other suitable method to detect the level of biomarker associated with the biological activity of the compound of interest.

Similarly, a competitive assay can be utilized to determine if a compound is tissue protective. In the competitive assay, a compound known to be tissue protective including, but not limited to, tissue protective cytokines such as those disclosed in U.S. patent application Ser. Nos. 10/188,905 and 10/185,841 (each of which is incorporated by reference herein in its entirety), can be attached to a suitable bio marker.

In a 96 well plate eight 1:2 serial dilutions of a known tissue protective compound/biomarker in suitable growth medium, and the same dilution series of the known tissue protective compound/biomarker and an excess of the compound of interest are plated. The final volume of each dilution should be about 100 μl. Once again, the BaF3 cells are seeded into the plates as disclosed supra and allowed to incubate. After an appropriate amount of time, the cells are washed and the plate is read on a fluorescent plate reader or by any other suitable method known in the art to detect the biomarker. If the readout of the plates and/or wells containing the known tissue protective compound/biomarker and compound of interest is less than the readout of the plates containing only the known tissue protective compound/biomarker then the compound of interest is tissue protective.

Many protein factors discovered to date, including all known cytokines, have exhibited activity in one or more factor-dependent cell proliferation assays, and hence these assays serve as a convenient confirmation of cytokine activity. The activity of a compound can be evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK. These cells are cultured in the presence or absence of a compound, and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, 1983, J. Immunol. Meth. 65:55-63, which is incorporated by reference herein in its entirety).

If a compound exhibits a tissue protective activity, one of ordinary skill in the art would recognize that it would be beneficial to verify the result using one of the neuroprotective and tissue protective assays known to those skilled in the art, such as, but not limited to, P-19 and PC-12 cell assays. Additionally, various in vivo models such as animal models related to spinal cord injury, ischemic stroke, peripheral nerve damage, heart, eyes, kidneys, etc. would be helpful in further characterizing the compound. Suitable in vitro and in vivo assays are disclosed in U.S. patent application Ser. Nos. 10/188,905 and 10/185,841, each of which is incorporated by reference herein in its entirety.

### 8. THERAPEUTIC USE

The above-noted compounds are useful as therapeutics for treatment, prevention, amelioration or management of various cancers or neoplastic diseases, symptoms thereof, i.e. cachexia, or the side effects associated with other treatments thereof.

The aforementioned method of treatment using the above-disclosed compounds may be useful generally for the prevention, therapeutic treatment, prophylactic treatment or management of various cancers or neoplastic disorders of the central nervous system, peripheral nervous system, gastrointestinal/digestive system, genitourinary system, gynecological, head and neck, hematological/blood, musculoskeletal/soft tissue, respiratory, and breast. Examples of use include, but are not limited to, protection against and repair of injury resulting from cancers or neoplastic disorders of the brain (astrocytoma, gliobastoma, glioma), spinal cord, pituitary gland, breast (Infiltrating, Pre-invasive, inflammatory cancers, Paget's Disease, Metastatic and Recurrent Breast Cancer), blood (Hodgkin's Disease, Leukemia, Multiple Myeloma, Lymphoma), Lymph node cancer, Lung (Adenocarcinoma, Oat Cell, Non-small Cell, Small Cell, Squamous Cell, Mesothelioma), skin (melanoma, basal cell, squamous cell, Kapsosi's Sarcoma), Bone Cancer (Ewing's Sarcoma, Osteosarcoma, Chondrosarcoma), head and neck (laryngeal, pharyngeal (nasal cavity & sinus cavity), and esophageal cancers), oral (jaw, salivary gland, throat, thyroid, tongue, and tonsil cancers), eye, gynecological (Cervical, Endrometrial, Fallopian, Ovarian, Uterine, Vaginal, and Vulvar), Genitourinary (Adrenal, bladder, kidney, penile, prostate, testicular, and urinary cancers), and gastrointestinal (appendix, bile duct (extrahepatic bile duct) colon, gallbladder, gastric, intestinal, colon, liver, pancreatic, rectal, and stomach cancers) as well as those listed below: (for a review of such disorders, see Fishman et al., 1985, Medicine, 2d Ed., J.B. Lippincott Co., Philadelphia): Leukemia: acute leukemia, acute lymphocytic leukemia, acute myelocytic leukemia, myeloblastic, promyelocytic, myelomonocytic, monocytic erythroleukemia, chronic leukemia, chronic myelocytic (granulocytic) leukemia, chronic lymphocytic leukemia, Polycythemia vera, Gastric carcinoma, Lymphoma (malignant and non-malignant): Hodgkin's disease, non-Hodgkin's disease, Multiple myeloma, Waldenstrom's macroglobulinemia, Heavy chain disease, Solid tumors sarcomas and carcinomas: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, oral squamous cell carcinoma, hepatocellular carcinoma, basal cell carcinoma, adenocarcinoma, sweat gland carcinoma, sebaceous gland carcinoma, papillary carcinoma, papillary adenocarcinomas: cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, cervix adenocarcinoma, uterine cancer, testicular tumor, lung carcinoma, small cell lung carcinoma, non-small cell lung adenocarcinoma, bladder carcinoma, epithelial carcinoma, glioma, malignant glioma, glioblastoma, multiforme astrocytic gliomas, medulloblastoma, craniopharyngioma, ependymoma, pinealoma, hemangioblastoma, acoustic neuroma, oligodendroglioma, meningioma, melanoma, neuroblastoma, retinoblastoma, etc.

In specific embodiments, cancer, malignancy or dysproliferative changes (such as metaplasias and dysplasias), or hyperproliferative disorders, are treatable or preventable in the ovary, breast, colon, lung, skin, pancreas, prostate, bladder, or uterus. In other specific embodiments, the cancer treatable or preventable by the administration of an effective amount of a compound is sarcoma, melanoma, or leukemia. In other specific embodiments, the cancer treatable or preventable by the administration of an effective amount of a compound is multiple myeloma, leukemia, a myelodysplastic syndrome or a myeloproliferative disorder. In another specific embodiment, the cancer treatable or preventable by the administration of an effective amount of a compound is a glioma.

In preferred embodiment, the method of treatment utilizing the compounds is useful for treating or preventing cancers including prostate (more preferably hormone-insensitive), Neuroblastoma, Lymphoma (preferably follicular or Diffuse Large B-cell), Breast (preferably Estrogen-receptor positive), Colorectal, Endometrial, Ovarian, Lymphoma (preferably non-Hodgkin's), Lung (preferably Small cell), or Testicular (preferably germ cell).

In another embodiment, the current method of treatment is useful for inhibiting the growth of a cell derived from a cancer or neoplasm such as prostate (more preferably hormone-insensitive), Neuroblastoma, Lymphoma (preferably follicular or Diffuse Large B-cell), Breast (preferably Estrogen-receptor positive), Colorectal, Endometrial, Ovarian, Lymphoma (preferably non-Hodgkin's), Lung (preferably Small cell), or Testicular (preferably germ cell).

In specific embodiments of the invention, the method of treatment of the Invention is useful for inhibiting the growth of a cell, said cell being derived from a cancer or neoplasm. As demonstrated in Examples 1 and 2, the compound administered in accordance with the current method of treatment were able to prevent implanted tumors from growing (Example 1 & 2).

In a further embodiment of the invention, the method of treatment of the current invention is useful for preventing, treating, ameliorating, or managing the symptoms associated with cancer or neoplastic disorders. In particular, the current method of treatment can be used to address the cachexia, wasting, associated with cancer. The above-noted compounds may be administered to offset the wasting associated with cancer in order to maintain the fat and lean mass of the patient, the appetite and activity levels of the patient and in general their sense of well being.

Further, the compound may be administered according to the current method to treat, ameliorate or manage various syndromes associated with various functional benign or cancerous tumors. Amongst those syndromes that may benefit from treatment with the compounds are Beckwith-Wiedmann Syndrome, SBLA Syndrome, Li-Fraumeni Syndrome, Familial Adenomatous Polyposis syndrome (Gardner Syndrome), Hereditary Nonpolyposis Colorectal Cancer, Turcot Syndrome, Cowden Syndrome, Carney Triad Syndrome, Multiple Endocrine Neoplasia Syndromes (Wermer (MEN-1), Sipple (MEN-2a, MEN-2b), Von Hipple-Lindau Syndrome, Cushing's Syndrome, Addison's Syndrome, Verner Morrison Syndrome, Zollinger-Ellison Syndrome, WDHA Syndrome, Pancreatic Cholera, Isaac's Syndrome, Rippling muscle syndrome, Stiffman syndrome, Paraneoplastic Ataxia, Yo Syndrome, Tr Syndrome, Hu Syndrome, CV-2 Syndrome, CRMP-5 Syndromes, Opsoclonus/Myoclonus, Ma Syndromes, Morvan's fibrillary chorea, Bannayan-Riley-Runalcaba Syndrome, Peutz-Jegher Syndrome, Muir-Tone Syndrome, Hirschsprung Disease, Lynch Syndrome, Lambert-Eaton Myastenic Syndrome, Myasthenia Gravis, Neuromyotonia, Paraneoplastic Cerebellat Degeneration, Paraneoplastic Limbic Encephalitis, Sweets Syndrome, Birt-Hogg-Dube Syndrome, Naevoid Basal Cell Carcinoma Syndrome, Generalized Basaloid Follicular, Hamartoma Syndrome, Bazex Syndrome, Brooke Spiegler Syndrome, Familial Cylindromatosis, Multiple Familial Trichoepitheliomas, Androgen Deprivation Syndrome, Therapy Related Myelodysplastic Syndrome, Somnolence Syndrome, Gulf War Syndrome, and Somatostatinoma Syndrome. The compounds may be used in accordance with the method of the current invention to address the above-noted syndromes. For example, the compounds may be administered to address hereditary syndromes such as Li Fraumeni, Hereditary Nonpolyposis Colorectal Cancer, Familial Adenomatous Polyposis, and Von Hippel-Lindau Syndrome by either delaying the onset of the neoplastic aspects of the disease, reducing the number of neoplastic growths associated with the syndrome, or in general enhancing the quality of life or the longevity of those patients afflicted with these conditions. The compounds may also be administered prophylactically to address syndromes related to certain treatment, chemotherapy or radiation therapy, of the neoplastic disorder or cancer, such as androgen deprivation syndrome, therapy related myelodysplastic syndrome or somnolence syndrome, in the hopes of preventing the syndromes or reducing the severity of the syndrome.

Further, the compound may be used to inhibit angiogenesis and growth promoting molecules by the matrix.

As mentioned above, these cancers and neoplastic disorders are merely illustrative of the range of disorders that can be addressed by the compounds used in the method of the current invention. Accordingly, this invention generally provides preventative, therapeutic, or prophylactic treatment of the consequences of cancers or neoplastic disorders.

Cancer or a neoplastic disease, including, but not limited to, a neoplasm, a tumor, a metastasis, or any disease or disorder characterized by uncontrolled cell growth, can be treated or prevented by administration of an effective amount of a Compound of the Invention. In one embodiment, a composition comprising an effective amount of one or more Compounds of the Invention, or a pharmaceutically acceptable salt thereof, is administered.

In certain embodiments, the invention encompasses methods for treating or preventing cancer or a neoplastic disease comprising administering to a patient need thereof an effective amount of a compound and another therapeutic agent. In one embodiment, the therapeutic agent is a chemotherapeutic agent including, but not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In one embodiment, the compound exerts its activity at the same time the other therapeutic agent exerts its activity. Other therapeutic agents are: Radiation: .gamma.-radiation, Alkylating agents Nitrogen mustards: cyclophosphamide, Ifosfamide trofosfamide, Chlorambucil, Nitrosoureas: carmustine (BCNU), Lomustine (CCNU), Alkylsulphonates busulfan, Treosulfan, Triazenes: Dacarbazine, Platinum containing compounds: Cisplatin carboplatin, Plant Alkaloids, Vinca alkaloids: vincristine, Vinblastine, Vindesine, Vinorelbine, Taxoids: paclitaxel, Docetaxol, DNA Topoisomerase Inhibitors Epipodophyllins: etoposide, Teniposide, Topotecan, 9-aminocamptothecin irinotecan (Campto®), crisnatol, Mytomycins: Mytomycin C, Mytomycin C Anti-metabolites, Anti-folates: DHFR inhibitors: methotrexate, Trimetrexate, IMP dehydrogenase Inhibitors: mycophenolic acid, Tiazofurin, Ribavirin EICAR, Ribonucleotide reductase Inhibitors: hydroxyurea deferoxamine, Pyrimidine analogs: Uracil analogs, 5-Fluorouracil, Floxuridine, Doxifluridine, Ratitrexed, Cytosine analogs cytarabine (ara C) Cytosine arabinoside fludarabine, Purine analogs: mercaptopurine, Thioguanine, Hormonal therapies Receptor antagonists: Anti-estrogens, Tamoxifen, Raloxifene megestrol, LHRH agonists: goscrclin, Leuprolide, acetate Anti-androgens: flutamide, bicalutamide, Retinoids/Deltoids Vitamin D3 analogs: EB 1089, CB 1093, KH 1060, Photodyamic therapies: vertoporfin (BPD-MA), Phthalocyanine photosensitizer, Pc4 Demethoxy-hypocrellin A (2BA-2-DMHA) Cytokines: Interferon-.alpha. Interferon-.gamma. Tumor necrosis factor Others: Isoprenylation inhibitors: Lovastatin Dopaminergic neurotoxins: 1-methyl-4-phenylpyridinium ion Cell cycle inhibitors: staurosporine, Actinomycins: Actinomycin D, Dactinomycin, Bleomycins: bleomycin A2, Bleomycin B2, Peplomycin, Anthracyclines: daunorubicin, Doxorubicin (adriamycin), Idarubicin, Epirubicin, Pirarubicin, Zorubicin, Mitoxantrone, MDR inhibitors: verapamil, Ca.sup.2+ ATPase inhibitors: thapsigargin, TNF-a inhibitors/thalidomide angiogenesis inhibitors 3-(3,4-dimethoxy-phenyl)-3-(1-oxo-1,3-dihydro-isoindol-2-yl)-propionamide (SelCIDs™) ImiDs™ Revimid™ Actimid™.

In other embodiments, the present methods for treating or preventing cancer further comprise administering radiation therapy. The cancer can be refractory or non-refractory. The compound can be administered to a patient that has undergone surgery as treatment for the cancer.

In a specific embodiment, compound can be administered to a patient that has undergone surgery as treatment for the cancer concurrently with chemotherapy or radiation therapy. In another specific embodiment, a chemotherapeutic agent or radiation therapy is administered prior or subsequent to administration of a compound, preferably at least an hour, five hours, 12 hours, a day, a week, a month, more preferably several months (e.g., up to three months).

The chemotherapeutic agent or radiation therapy administered concurrently with, or prior or subsequent to, the administration of a compound can be accomplished by any method known in the art. The chemotherapeutic agents are preferably administered in a series of sessions, any one or a combination of the chemotherapeutic agents listed above can be administered. With respect to radiation therapy, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, may also be administered to expose tissues to radiation.

Additionally, the invention provides methods of treatment of cancer or neoplastic disease with a compound as an alternative to chemotherapy or radiation therapy where the chemotherapy or the radiation therapy has proven or may prove too toxic, e.g., results in unacceptable or unbearable side effects, for the patient being treated. Alternatively, the invention provides methods of treatment wherein the compound is administered prior to, simultaneously with or following treatment with chemotherapy or radiation in an effort to prevent or ameliorate the toxic side effects of the treatment method. As demonstrated in Example 2, the compounds administered in accordance with the current method are able to ameliorate the side-effects of cis-platinum a known chemotherapeutic. The patient being treated can, optionally, be treated with other cancer treatments such as surgery, radiation therapy or chemotherapy, depending on which treatment is found to be acceptable or bearable.

In one embodiment, the method of the current invention provides that a pharmaceutical composition comprising a compound can be administered systemically to protect or enhance the target cells, tissue or organ. Such administration may be parenterally, via inhalation, or transmucosally, e.g., orally, nasally, rectally, intravaginally, sublingually, ocularly, submucosally or transdermally. Preferably, administration is parenteral, e.g., via intravenous or intraperitoneal injection, and also including, but is not limited to, intra-arterial, intramuscular, intradermal and subcutaneous administration.

For other routes of administration, such as by use of a perfusate, injection into an organ, or other local administration, a pharmaceutical composition will be provided which results in similar levels of a compound as described above. A level of about 15 pM-30 nM is preferred.

The pharmaceutical compositions of the invention may comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized foreign pharmacopeia for use in animals, and more particularly in humans. The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc. Examples of suitable pharmaceutical carriers are described in “Remington's Pharmaceutical Sciences” by E. W. Martin, hereby incorporated by reference herein in its entirety. Such compositions will contain a therapeutically effective amount of the compound, preferably in purified form, together with a suitable amount of carrier so as to provide the form for proper administration to the patient. The formulation should suit the mode of administration.

Formulations for increasing transmucosal adsorption of compounds such as long acting compounds are also contemplated by the current invention. Pharmaceutical compositions adapted for oral administration may be provided as capsules or tablets; as powders or granules; as solutions, syrups or suspensions (in aqueous or non-aqueous liquids); as edible foams or whips; or as emulsions. Tablets or hard gelatine capsules may comprise lactose, starch or derivatives thereof, magnesium stearate, sodium saccharine, cellulose, magnesium carbonate, stearic acid or salts thereof. Soft gelatine capsules may comprise vegetable oils, waxes, fats, semi-solid, or liquid polyols etc. Solutions and syrups may comprise water, polyols and sugars.

An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Pharmaceutical compositions adapted for transdermal administration may be provided as discrete patches intended to remain in intimate contact with the epidermis of the recipient for a prolonged period of time. Pharmaceutical compositions adapted for topical administration may be provided as ointments, creams, suspensions, lotions, powders, solutions, pastes, gels, sprays, aerosols or oils. For topical administration to the skin, mouth, eye or other external tissues a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base. Pharmaceutical compositions adapted for topical administration to the eye include eye drops. In these compositions, the active ingredient can be dissolved or suspended in a suitable carrier, e.g., in an aqueous solvent. Pharmaceutical compositions adapted for topical administration in the mouth include lozenges, pastilles and mouthwashes.

Pharmaceutical compositions adapted for nasal and pulmonary administration may comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers, e.g., nasal sprays or nasal drops. Alternatively, inhalation of compounds directly into the lungs may be accomplished by inhalation deeply or installation through a mouthpiece into the oropharynx. These compositions may comprise aqueous or oil solutions of the active ingredient. Compositions for administration by inhalation may be supplied in specially adapted devices including, but not limited to, pressurized aerosols, nebulizers or insufflators, which can be constructed so as to provide predetermined dosages of the active ingredient. In a preferred embodiment, pharmaceutical compositions of the invention are administered into the nasal cavity directly or into the lungs via the nasal cavity or oropharynx.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for parenteral administration include aqueous and non-aqueous sterile injectable solutions or suspensions, which may contain antioxidants, buffers, bacteriostats and solutes that render the compositions substantially isotonic with the blood of an intended recipient. Other components that may be present in such compositions include water, alcohols, polyols, glycerine and vegetable oils, for example. Compositions adapted for parenteral administration may be presented in unit-dose or multi-dose containers, for example sealed ampules and vials, and may be stored in a freeze-dried (lyophilized) condition requiring only the addition of a sterile liquid carrier, e.g., sterile saline solution for injections, immediately prior to use. Extemporaneous injection solutions and suspensions may be prepared from sterile powders, granules and tablets. In one embodiment, an autoinjector comprising an injectable solution of a compound may be provided for emergency use by ambulances, emergency rooms, and battlefield situations, and even for self-administration in a domestic setting, particularly where the possibility of traumatic amputation may occur, such as by imprudent use of a lawn mower. The likelihood that cells and tissues in a severed foot or toe will survive after reattachment may be increased by administering a compound to multiple sites in the severed part as soon as practicable, even before the arrival of medical personnel on site, or arrival of the afflicted individual with severed toe in tow at the emergency room.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water-free concentrate in a hermetically-sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile saline can be provided so that the ingredients may be mixed prior to administration.

Suppositories generally contain active ingredient in the range of 0.5% to 10% by weight; oral formulations preferably contain 10% to 95% active ingredient.

A perfusate composition may be provided for use in transplanted organ baths, for in situ perfusion, or for administration to the vasculature of an organ donor prior to organ harvesting. Such pharmaceutical compositions may comprise levels of compounds, or a form of compounds not suitable for acute or chronic, local or systemic administration to an individual, but will serve the functions intended herein in a cadaver, organ bath, organ perfusate, or in situ perfusate prior to removing or reducing the levels of the compound contained therein before exposing or returning the treated organ or tissue to regular circulation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, for example, a compound can be delivered in a controlled-release system. For example, the compound may be administered using intravenous infusion, an implantable osmotic pump, a transdermal patch, liposomes, or other modes of administration. In one embodiment, a pump may be used (see Langer, supra; Sefton, 1987, CRC Crit. Ref. Biomed. Eng. 14:201; Buchwald et al., 1980, Surgery 88:507; Saudek et al., 1989, N. Engl. J. Med. 321:574, each of which is incorporated by reference herein in its entirety). In another embodiment, the compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); WO 91/04014; U.S. Pat. No. 4,704,355; Lopez-Berestein, ibid., pp. 317-327; see generally ibid.). In another embodiment, polymeric materials can be used (see Medical Applications of Controlled Release, Langer and Wise (eds.), CRC Press: Boca Raton, Fla., 1974; Controlled Drug Bioavailability, Drug Product Design and Performance, Smolen and Ball (eds.), Wiley: New York (1984); Ranger and Peppas, J. Macromol. Sci. Rev. Macromol. Chem. 23:61, 1953; see also Levy et al., 1985, Science 228:190; During et al., 1989, Ann. Neurol. 25:351; Howard et al., 1989, J. Neurosurg. 71:105, (each of which is incorporated by reference herein in its entirety).

In yet another embodiment, a controlled release system can be placed in proximity of the therapeutic target, i.e., the target cells, tissue or organ, thus requiring only a fraction of the systemic dose (see, e.g., Goodson, pp. 115-138 in Medical Applications of Controlled Release, vol. 2, supra, 1984, which is incorporated by reference herein in its entirety). Other controlled release systems are discussed in the review by Langer (1990, Science 249:1527-1533, which is incorporated by reference herein in its entirety).

In another embodiment, compound, as properly formulated, can be administered by nasal, oral, rectal, vaginal, ocular, transdermal, parenteral or sublingual administration.

In a specific embodiment, it may be desirable to administer a compound of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as silastic membranes, or fibers. A non-limiting example of such an embodiment would be a stent or other scaffolding coated with a compound of the present invention implanted in a portion of the vasculature, duct, etc. affected by cancer or a neoplastic disorder.

Selection of the preferred effective dose will be readily determinable by a skilled artisan based upon considering several factors, which will be known to one of ordinary skill in the art. Such factors include the particular form of compound, and its pharmacokinetic parameters such as bioavailability, metabolism, half-life, etc., which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether administration is acute or chronic, concomitant medications, and other factors well known to affect the efficacy of administered pharmaceutical agents. Thus the precise dosage should be decided according to the judgment of the practitioner and each patient's circumstances, e.g., depending upon the condition and the immune status of the individual patient, and according to standard clinical techniques.

In another aspect of the present invention, a pharmaceutical composition according to the present invention may include a compound in a formulation with at least one small molecule that exhibits tissue protective functionality. Suitable small molecules include, but are not limited to, steroids (e.g., lazaroids and glucocorticoids), antioxidants (e.g., coenzyme Q₁₀, alpha lipoic acid, and NADH), anticatabolic enzymes (e.g., glutathione peroxidase, superoxide dimutase, catalase, synthetic catalytic scavengers, as well as mimetics), indole derivatives (e.g., indoleamines, carbazoles, and carbolines), nitric acid neutralizing agents, adenosine/adenosine agonists, phytochemicals (flavanoids), herbal extracts (ginko biloba and turmeric), vitamins (vitamins A, E, and C), oxidase electron acceptor inhibitors (e.g., xanthine oxidase electron inhibitors), minerals (e.g., copper, zinc, and magnesium), non-steriodal anti-inflammatory drugs (e.g., aspirin, naproxen, and ibuprofen), and combinations thereof. Additionally agents including, but not limited to, anti-inflammatory agents (e.g., corticosteroids, prednisone and hydrocortisone), glucocorticoids, steroids, non-steriodal anti-inflammatory drugs (e.g., aspirin, ibuprofen, diclofenac, and COX-2 inhibitors), beta-agonists, anticholinergic agents and methyl xanthines), immunomodulatory agents (e.g., small organic molecules, T cell receptor modulators, cytokine receptor modulators, T-cell depleting agents, cytokine antagonists, monokine antagonists, lymphocyte inhibitors, or anti-cancer agents), gold injections, sulphasalazine, penicillamine, anti-angiogenic agents (e.g., angiostatin), TNF-α antagonists (e.g., anti-TNFα antibodies), and endostatin), dapsone, psoralens (e.g., methoxalen and trioxsalen), anti-malarial agents (e.g., hydroxychloroquine), anti-viral agents, and antibiotics (e.g., erythromycin and penicillin) may be used in conjunction with the current pharmaceutical compositions.

In another aspect of the invention, a perfusate or perfusion solution is provided for perfusion of afflicted tissue samples and or organs during ex vivo procedures such as bench surgical procedures, in which an organ may be removed, and while ex vivo, resected, repaired, or otherwise manipulated, such as for tumor removal, and then returned to the original location. In one embodiment, the perfusion solution is the University of Wisconsin (UW) solution (U.S. Pat. No. 4,798,824, hereby incorporated by reference herein in its entirety) which contains from about 1 to about 25 U/ml (10 ng=1U) of compound, 5% hydroxyethyl starch (having a molecular weight of from about 200,000 to about 300,000 and substantially free of ethylene glycol, ethylene chlorohydrin, sodium chloride and acetone); 25 mM KH₂PO₄; 3 mM glutathione; 5 mM adenosine; 10 mM glucose; 10 mM HEPES buffer; 5 mM magnesium gluconate; 1.5 mM CaCl₂; 105 mM sodium gluconate; 200,000 units penicillin; 40 units insulin; 16 mg dexamethasone; 12 mg Phenol Red; and has a pH of 7.4-7.5 and an osmolality of about 320 mOsm/l. This particular perfusate is merely illustrative of a number of such solutions that can be adapted for the present use by inclusion of an effective amount of a compound. In a further embodiment, the perfusate solution contains from about 1 to about 500 ng/ml of a compound, or from about 40 to about 320 ng/ml compound. As mentioned above, any form of compound can be used in this aspect of the invention.

While the preferred recipient of a compound for the purposes herein throughout is a human, the methods herein apply equally to other mammals, particularly domesticated animals, livestock, companion, and zoo animals. However, the invention is not so limiting and the benefits can be applied to any mammal.

In further aspects of the ex-vivo invention, any compound such as but not limited to the ones described above may be employed.

In another aspect of the invention, methods and compositions for preventing, treating or managing cancerous cells, tissues or organs which are not isolated from the vasculature by an endothelial cell barrier are provided by exposing the cells, tissue or organs directly to a pharmaceutical composition comprising a compound, or administering or contacting a pharmaceutical composition containing a compound to the vasculature of the tissue or organ. Enhanced activity of responsive cells in the treated tissue or organ is responsible for the positive effects exerted.

Similar to other tissue protective compounds based on erythropoietin, it is possible that the compounds of the present invention may be transported from the luminal surface to the basement membrane surface of endothelial cells of the capillaries of organs with endothelial cell tight junctions, including, for example, the brain, retina, and testis. Thus, responsive cells across the barrier may be susceptible targets for the beneficial effects of compounds, and others cell types or tissues or organs that contain and depend in whole or in part on responsive cells therein may be targets for the methods of the invention. While not wishing to be bound by any particular theory, after transcytosis of the compound may interact with an tissue-protective receptor on a responsive cell, for example, neuronal, eye (e.g., retinal), adipose, connective, hair, tooth, mucosal, pancreatic, endocrine, aural, epithelial, skin, muscle, heart, lung, liver, kidney, small intestine, adrenal (e.g. adrenal cortex, adrenal medulla), capillary, endothelial, testes, ovary, or endometrial cell, and receptor binding can initiate a signal transduction cascade resulting in the activation of a gene expression program within the responsive cell or tissue, resulting in the protection of the cell or tissue, or organ, from damage, such as by toxins, chemotherapeutic agents, radiation therapy, hypoxia, etc. In another embodiment, the compound can be cross-linked to a compound that can cross the barrier, such as carbamoylated erythropoietin, to be transported across the barrier in accordance with the teaching of PCT Application No. PCT/US01/49479, U.S. patent application Ser. Nos. 10/188,905 and 10/185,841, incorporated herein by reference. Thus, methods for protecting responsive cell-containing tissue from injury or hypoxic stress, and enhancing the function of such tissue are described in detail herein below.

In the practice of one embodiment of the invention, a mammalian patient is undergoing systemic chemotherapy for cancer treatment, including radiation therapy, which commonly has adverse effects such as nerve, lung, heart, ovarian or testicular damage. Administration of a pharmaceutical composition comprising a compound as described above is performed prior to and during chemotherapy and/or radiation therapy, to protect various tissues and organs from damage by the chemotherapeutic agent, such as to protect the testes. Treatment may be continued until circulating levels of the chemotherapeutic agent have fallen below a level of potential danger to the mammalian body.

In the practice of another embodiment of the invention, various organs are planned to be harvested from a victim of an automobile accident for transplant into a number of recipients, some of which required transport for an extended distance and period of time. Prior to organ harvesting, the donor is infused with a pharmaceutical composition comprising compounds as described herein. Harvested organs for shipment are perfused with a perfusate containing compounds as described herein, and stored in a bath comprising compounds. Certain organs are continuously perfused with a pulsatile perfusion device, utilizing a perfusate containing compounds in accordance with the present invention. Minimal deterioration of organ function occurs during the transport and upon implant and reperfusion of the organs in situ.

In another embodiment of the present invention, a participant in a hazardous activity, one could take a dose of a pharmaceutical composition containing a compound sufficient to either prevent (i.e. delaying the onset of, inhibiting, or stopping), protect against, or mitigate the damage resulting from possible carcinogens. In particular, this method of treatment may have application in various professions exposed to known carcinogens such as miners, chemical manufacturers, researchers, etc. as well as military personnel (soldiers, paratroopers), emergency personnel (police, fire, EMS, and disaster relief personnel) and construction workers who may be exposed to carcinogens in the performance of their duties. Additionally, the prophylactic use of compounds is contemplated where the patient has an inherited predisposition for the development of cancer or neoplastic disorders.

In the foregoing examples in which a compound is used for ex-vivo applications, or for in vivo applications to treat cancers or neoplastic disorders, the invention provides a pharmaceutical composition in dosage unit form adapted for prevention, treatment or management of the cancer or neoplastic disorder which comprises an amount within the range from about 0.01 pg to 30 mg, 0.5 pg to 20 mg, 1 pg to 10 mg, 500 pg to 5 mg, 1 ng to 5 mg, 500 ng to 5 mg, 1 μg to 5 mg, 500 μg to 5 mg, or 1 mg to 5 mg of a compound, and a pharmaceutically acceptable carrier. In a preferred embodiment, the amount of compound is within the range from about 0.5 pg to 1 mg. In a preferred embodiment, the formulation contains compounds that are non-erythropoietic.

In a further aspect of the invention, administration of compounds may be used to restore cognitive function in mammals having undergone whole brain irradiation. After a delay of either 5 days or 30 days, administration of compounds should be able to restore function as compared to placebo-treated mammals, indicating the ability of the compound to regenerate or restore brain activity. Thus, the invention is also directed to the use of compounds for the preparation of a pharmaceutical composition for the treatment of brain trauma and other cognitive dysfunctions, including treatment well after the injury (e.g. three days, five days, a week, a month, or longer). The invention is also directed to a method for the treatment of cognitive dysfunction following injury by administering an effective amount of compounds. Any compound as described herein may be used for this aspect of the invention.

Furthermore, this restorative aspect of the invention is directed to the use of any compounds herein for the preparation of a pharmaceutical composition for the restoration of cellular, tissue or organ dysfunction, wherein treatment is initiated after, and well after, the initial insult responsible for the dysfunction. Moreover, treatment using compounds of the invention can span the course of the disease or condition during the acute phase as well as a chronic phase.

A compound of the invention may be administered systemically at a dosage between about 1 ng and about 5000 μg/kg body weight, preferably about 5-500 μg/kg-body weight, most preferably about 10-300 μg/kg-body weight, per administration. This effective dose should be sufficient to achieve serum levels of compounds greater than about 80, 120, or 160 ng/ml of serum after administration. Such serum levels may be achieved at about 1, 2, 3, 4, 5, 6, 7, 8, 9, or 10 hours post-administration. Such dosages may be repeated as necessary. For example, administration may be repeated daily, as long as clinically necessary, or after an appropriate interval, e.g., every 1 to 12 weeks, preferably, every 1 to 3 weeks. In one embodiment, the effective amount of compound and a pharmaceutically acceptable carrier may be packaged in a single dose vial or other container. In another embodiment, the compounds, which are capable of exerting the activities described herein but not causing an increase in hemoglobin concentration or hematocrit, are used. Such compounds are preferred in instances wherein the methods of the present invention are intended to be provided chronically.

### EXAMPLES

### Example 1

### Cortical Implant

A cortical tumor implant study was conducted in accordance with the protocol of Lampson et al. Cancer Res. 53:176-82:1993 to determine the effect of CEPO on the growth of the tumor.

In accordance with the protocol, male CD fisher rats were anesthetized and their right scalp was shaved and washed with disinfectant. A small incision was then made in the scalp overlying the right temporal cerebral cortex. A 1 mm hole was drilled through the calvarium, without puncturing the dura matter. Under aseptic conditions, living 10,000 9L/LacZ gliosarcoma cells in 5 μl was slowly injected through a 26 gauge needle attached to a precision Hamilton syringe. Bone wax was then applied to the trephanation site, the skin was sutured and a prophylactic dose of antibiotic was administered i.p. Control rats were provided with saline injections i.p. daily. One group of rats was treated with CEPO (10 μg/kg i.v.) and another group was treated with rhEPO (10 μg/kg i.v.) daily for a period of 21 days. At 21 days following implantation, the animal was anesthetized and the brain perfused-fixed with 2% paraformaldehyde. The brain was then removed and cut on a brain matrix device into coronal sections 1 mm thick and the extent of tumor mass was determined by planimetric methodology.

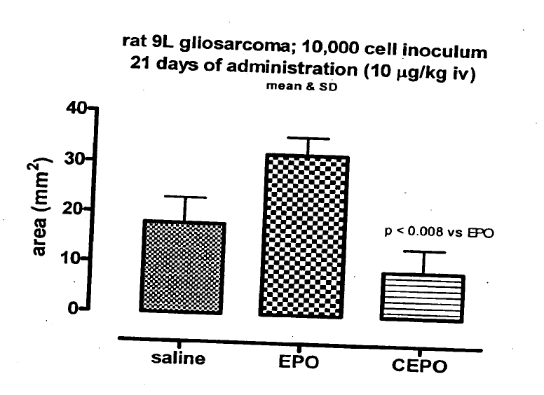

FIG. 1 demonstrates that CEPO inhibited the growth of the cortical tumor in this model. Whereas the relative mass of the tumor in the saline treated rats grew to nearly 20 mm², the tumors in the CEPO treated rats experienced about half the growth @ 10 mm². Surprisingly, the tumor in the rhEPO treated animals grew to a greater relative mass than the tumors in the saline treated rats.

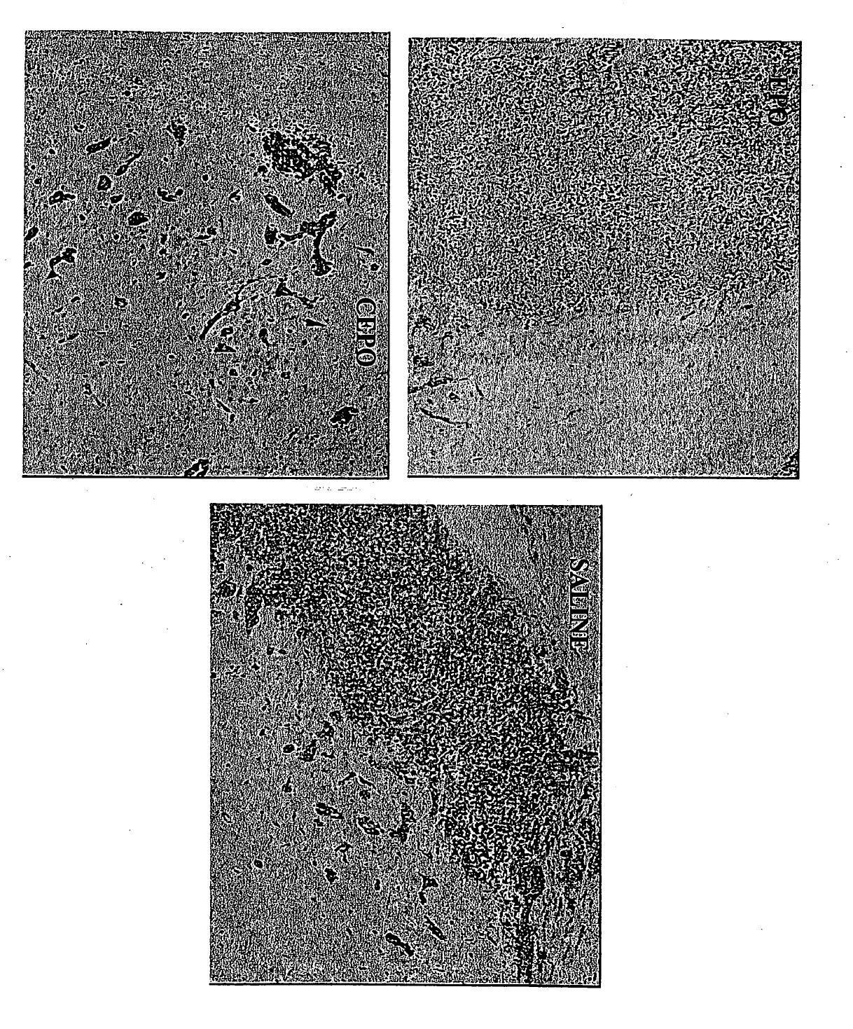

FIG. 2 shows the extension and spreading of the glioma within the cerebral parenchyma of the rats treated with rhEPO, Saline, and CEPO. Although both the rhEPO and Saline treated rats showed large masses of glioma, the CEPO treated rats show isolates areas of glioma.

### Example 2

### (Prophetic) Treatment in Cancer Cachexia

The ability of the compounds to address cachexia symptomatic of cancer may be verified by the following protocol.

Rats weighing about 200 g would be inoculated intraperitoneally with 10⁸ AH-130 Hepatoma cells. Simultaneously, one group of rats would be treated with a compound of interest such as, CEPO, at a dose that would be in a therapeutically effective range, about 0.10 to 20 μg/kd/d. A second group would be treated with a placebo. On day 16, the rats would be sacrificed. The food intake and locomotor activity of the subject rats would be assessed before inoculation and on day 11. The weight and body composition would be evaluated by NMR-scan on day 0 and day 16 after sacrifice.

In comparison to the placebo treated rats, one would expect those treated with a compound in accordance with the method of the current invention to exhibit less fat and lean mass wasting. In addition, the food intake and locomotor activity of the compound treated rats would be improved.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein are incorporated by reference herein in their entireties for all purposes.

1. A method for preventing, treating, ameliorating or managing cancer or a neoplastic disorder in a subject in need thereof by administering to the subject an effective amount of a chemically modified erythropoietin selected from the group consisting of i) an erythropoietin that lacks sialic acid moieties, ii) an erythropoietin having at least no sialic acid moieties; iii) an erythropoietin having at least no N-linked or no O-linked carbohydrates; iv) an erythropoietin having at least a reduced carbohydrate content by virtue of treatment of native erythropoietin with at least one glycosidase; v) an erythropoietin having at least one or more oxidized carbohydrates; vi) an erythropoietin having at least one or more oxidized carbohydrates and is chemically reduced; vii) an erythropoietin having at least one or more modified arginine residues; viii) an erythropoietin having at least one or more modified lysine residues or a modification of the N-terminal amino group of the erythropoietin molecule; ix) an erythropoietin having at least a modified tyrosine residue; x) an erythropoietin having at least a modified aspartic acid or a glutamic acid residue; xi) an erythropoietin having at least a modified tryptophan residue; xii) an erythropoietin having at least one amino group removed; xiii) an erythropoietin having at least an opening of at least one of the cystine linkages in the erythropoietin molecule; or xiv) a truncated erythropoietin.

2. The method of claim 1, wherein the chemically modified erythropoietin lacks erythropoietin's erythropoietic effects.

3. The method of claim 1, wherein the chemically modified erythropoietin comprises carbamoylated erythropoietin.

4. A method for preventing, treating, ameliorating or managing cancer or a neoplastic disorder in a subject in need thereof by administering to the subject an effective amount of a mutated erythropoietin selected from the group consisting of one or more of the following mutations C7S, R10I, V11S, L12A, E13A, R14A, R14B, R14E, R14Q, Y15A, Y15F, Y15I, K20A, K20E, E21A, C29S, C29Y, C33S, C33Y, P42N, T44I, K45A, K45D, V46A, N47A, F48A, F48I, Y49A, Y49S, W51F, W51N, Q59N, E62T, L67S, L70A, D96R, K97D, S100R, S100E, S100A, S100T, G101A, G101I, L102A, R103A, S104A, S104I, L105A, T106A, T106I, T107A, T107L, L108K, L108A, S126A, F142I, R143A, S146A, N147K, N147A, F148Y, L149A, R150A, G151A, K152A, L153A, L155A, C160S, I6A, C7A, B13A, N24K, A30N, H32T, N38K, N83K, P42A, D43A, K52A, K97A, K116A, T132A, I133A, T134A, K140A, P148A, R150B, G151A, K152W, K154A, G158A, C161A, and/or R162A.

5. The method of claim 4, wherein the mutated erythropoietin lacks erythropoietin's erythropoietic effects.

6. The method of claim 1, wherein said cancer is infiltrating breast cancer, pre-invasive breast cancer, inflammatory breast cancer, Paget's disease, metastatic breast cancer, recurrent breast cancer, appendix cancer, bile duct cancer, extrahepatic bile duct cancer, colon cancer, esophageal cancer, gallbladder cancer, gastric cancer, intestinal cancer, liver cancer, pancreatic cancer, rectal cancer, stomach cancer, adrenal cancer, bladder cancer, kidney cancer, penile cancer, prostate cancer, testicular cancer, urinary cancer, cervical cancer, endometrial cancer, fallopian tube cancer, ovarian cancer, uterine cancer, vaginal cancer, vulvar cancer, eye cancer, head and neck cancer, jaw cancer, laryngeal cancer, pharyngeal cancer, oral cancer, nasal cavity cancer, salivary gland cancer, sinus cancer, throat cancer, thyroid cancer, tongue cancer, tonsil cancer, Hodgkin's disease, leukemia, acute lymphocytic leukemia, acute granulocytic leukemia, acute myelogenous leukemia, chronic lymphatic leukemia, chronic myelogenous leukemia, multiple myeloma, lymphoma, b-cell lymphoma, lymph node cancer, bone cancer, osteosarcoma, melanoma, skin cancer, basal cell cancer, squamous cell cancer, sarcoma, Ewing's sarcoma, Kaposi's sarcoma, brain cancer, astrocytoma, glioblastoma, glioma, pituitary gland cancer, spinal cord cancer, lung cancer, adenocarcinoma, oat cell cancer, non-small cell lung cancer, small cell lung cancer, squamous cell cancer, or mesothelioma.

7-8. (canceled)

9. The method of claim 4, wherein said cancer is infiltrating breast cancer, pre-invasive breast cancer, inflammatory breast cancer, Paget's disease, metastatic breast cancer, recurrent breast cancer, appendix cancer, bile duct cancer, extrahepatic bile duct cancer, colon cancer, esophageal cancer, gallbladder cancer, gastric cancer, intestinal cancer, liver cancer, pancreatic cancer, rectal cancer, stomach cancer, adrenal cancer, bladder cancer, kidney cancer, penile cancer, prostate cancer, testicular cancer, urinary cancer, cervical cancer, endometrial cancer, fallopian tube cancer, ovarian cancer, uterine cancer, vaginal cancer, vulvar cancer, eye cancer, head and neck cancer, jaw cancer, laryngeal cancer, pharyngeal cancer, oral cancer, nasal cavity cancer, salivary gland cancer, sinus cancer, throat cancer, thyroid cancer, tongue cancer, tonsil cancer, Hodgkin's disease, leukemia, acute lymphocytic leukemia, acute granulocytic leukemia, acute myelogenous leukemia, chronic lymphatic leukemia, chronic myelogenous leukemia, multiple myeloma, lymphoma, b-cell lymphoma, lymph node cancer, bone cancer, osteosarcoma, melanoma, skin cancer, basal cell cancer, squamous cell cancer, sarcoma, Ewing's sarcoma, Kaposi's sarcoma, brain cancer, astrocytoma, glioblastoma, glioma, pituitary gland cancer, spinal cord cancer, lung cancer, adenocarcinoma, oat cell cancer, non-small cell lung cancer, small cell lung cancer, squamous cell cancer, or mesothelioma.
